SEQUENCE LISTING (25 sequences)

SEQ ID NO: 1 - protein, 25 aa, Artificial Sequence
  Synthetic
  MISC_FEATURE (1)..(10): Optionally absent
  MISC_FEATURE (15)..(15): Xaa is E or Q
  MISC_FEATURE (16)..(25): Optionally absent
ATTAGILATLSHCIXLMVKREDSWQ

SEQ ID NO: 2 - protein, 10 aa, Artificial Sequence
  Synthetic
ATTAGILATL

SEQ ID NO: 3 - protein, 10 aa, Artificial Sequence
  Synthetic
LMVKREDSWQ

SEQ ID NO: 4 - protein, 5 aa, Artificial Sequence
  Synthetic
SHCIE

SEQ ID NO: 5 - protein, 5 aa, Artificial Sequence
  Synthetic
SHCIQ

SEQ ID NO: 6 - protein, 15 aa, Artificial Sequence
  Synthetic
ILATLSHCIELMVKR

SEQ ID NO: 7 - protein, 15 aa, Artificial Sequence
  Synthetic
ILATLSHCIQLMVKR

SEQ ID NO: 8 - protein, 14 aa, Artificial Sequence
  Synthetic
LATLSHCIELMVKR

SEQ ID NO: 9 - protein, 265 aa, Artificial Sequence
  Synthetic
RDEVIGILKAEKMDLALLEAQYGFVTPKKVLEALQRDAFQAKSTPWQEDIYEKPMNELDKVVEKHKESYRRILGQLLVAEKSRRQTILELEEEKRKHKEYMEKSDEFICLLEQECERLKKLIDQEIKSQEEKEQEKEKRVTTLKEELTKLKSFALMVVDEQQRLTAQLTLQRQKIQELTTNAKETHTKLALAEARVQEEEQKATRLEKELQTQTTKFHQDQDTIMAKLTNEDSQNRQLQQKLAALSRQIDELEETNRSLRKAEEE

SEQ ID NO: 10 - protein, 624 aa, Homo sapiens
MSDNQSWNSSGSEEDPETESGPPVERCGVLSKWTNYIHGWQDRWVVLKNNALSYYKSEDETEYGCRGSICLSKAVITPHDFDECRFDISVNDSVWYLRAQDPDHRQQWIDAIEQHKTESGYGSESSLRRHGSMVSLVSGASGYSATSTSSFKKGHSLREKLAEMETFRDILCRQVDTLQKYFDACADAVSKDELQRDKVVEDDEDDFPTTRSDGDFLHSTNGNKEKLFPHVTPKGINGIDFKGEAITFKATTAGILATLSHCIELMVKREDSWQKRLDKETEKKRRTEEAYKNAMTELKKKSHFGGPDYEEGPNSLINEEEFFDAVEAALDRQDKIEEQSQSEKVRLHWPTSLPSGDAFSSVGTHRFVQKPYSRSSSMSSIDLVSASDDVHRFSSQVEEMVQNHMTYSLQDVGGDANWQLVVEEGEMKVYRREVEENGIVLDPLKATHAVKGVTGHEVCNYFWNVDVRNDWETTIENFHVVETLADNAIIIYQTHKRVWPASQRDVLYLSVIRKIPALTENDPETWIVCNFSVDHDSAPLNNRCVRAKINVAMICQTLVSPPEGNQEISRDNILCKITYVANVNPGGWAPASVLRAVAKREYPKFLKRFTSYVQEKTAGKPILF

SEQ ID NO: 11 - protein, 598 aa, Homo sapiens
MSDNQSWNSSGSEEDPETESGPPVERCGVLSKWTNYIHGWQDRWVVLKNNALSYYKSEDETEYGCRGSICLSKAVITPHDFDECRFDISVNDSVWYLRAQDPDHRQQWIDAIEQHKTESGYGSESSLRRHGSMVSLVSGASGYSATSTSSFKKGHSLREKLAEMETFRDILCRQVDTLQKYFDACADAVSKDELQRDKVVEDDEDDFPTTRSDGDFLHSTNGNKEKLFPHVTPKGINGIDFKGEAITFKATTAGILATLSHCIELMVKREDSWQKRLDKETEKKRRTEEAYKNAMTELKKKSHFGGPDYEEGPNSLINEEEFFDAVEAALDRQDKIEEQSQSEKVRLHWPTSLPSGDAFSSVGTHRFVQKVEEMVQNHMTYSLQDVGGDANWQLVVEEGEMKVYRREVEENGIVLDPLKATHAVKGVTGHEVCNYFWNVDVRNDWETTIENFHVVETLADNAIIIYQTHKRVWPASQRDVLYLSVIRKIPALTENDPETWIVCNFSVDHDSAPLNNRCVRAKINVAMICQTLVSPPEGNQEISRDNILCKITYVANVNPGGWAPASVLRAVAKREYPKFLKRFTSYVQEKTAGKPILF

SEQ ID NO: 12 - protein, 707 aa, Homo sapiens
TAAAADGWKGRLPSPLVLLPRSARCQARRRRGGRTSSLLLLPPTPERALFASPSPDPSPRGLGASSGAAEGAGAGLLLGCRASMSDNQSWNSSGSEEDPETESGPPVERCGVLSKWTNYIHGWQDRWVVLKNNALSYYKSEDETEYGCRGSICLSKAVITPHDFDECRFDISVNDSVWYLRAQDPDHRQQWIDAIEQHKTESGYGSESSLRRHGSMVSLVSGASGYSATSTSSFKKGHSLREKLAEMETFRDILCRQVDTLQKYFDACADAVSKDELQRDKVVEDDEDDFPTTRSDGDFLHSTNGNKEKLFPHVTPKGINGIDFKGEAITFKATTAGILATLSHCIELMVKREDSWQKRLDKETEKKRRTEEAYKNAMTELKKKSHFGGPDYEEGPNSLINEEEFFDAVEAALDRQDKIEEQSQSEKVRLHWPTSLPSGDAFSSVGTHRFVQKPYSRSSSMSSIDLVSASDDVHRFSSQVEEMVQNHMTYSLQDVGGDANWQLVVEEGEMKVYRREVEENGIVLDPLKATHAVKGVTGHEVCNYFWNVDVRNDWETTIENFHVVETLADNAIIIYQTHKRVWPASQRDVLYLSVIRKIPALTENDPETWIVCNFSVDHDSAPLNNRCVRAKINVAMICQTLVSPPEGNQEISRDNILCKITYVANVNPGGWAPASVLRAVAKREYPKFLKRFTSYVQEKTAGKPILF

SEQ ID NO: 13 - skipped

SEQ ID NO: 14 - protein, 764 aa, Homo sapiens
MRSRGSDTEGSAQKKFPRHTKGHSFQGPKNMKHRQQDKDSPSESDVILPCPKAEKPHSGNGHQAEDLSRDDLLFLLSILEGELQARDEVIGILKAEKMDLALLEAQYGFVTPKKVLEALQRDAFQAKSTPWQEDIYEKPMNELDKVVEKHKESYRRILGQLLVAEKSRRQTILELEEEKRKHKEYMEKSDEFICLLEQECERLKKLIDQEIKSQEEKEQEKEKRVTTLKEELTKLKSFALMVVDEQQRLTAQLTLQRQKIQELTTNAKETHTKLALAEARVQEEEQKATRLEKELQTQTTKFHQDQDTIMAKLTNEDSQNRQLQQKLAALSRQIDELEETNRSLRKAEEELQDIKEKISKGEYGNAGIMAEVEELRKRVLDMEGKDEELIKMEEQCRDLNKRLERETLQSKDFKLEVEKLSKRIMALEKLEDAFNKSKQECYSLKCNLEKERMTTKQLSQELESLKVRIKELEAIESRLEKTEFTLKEDLTKLKTLTVMFVDERKTMSEKLKKTEDKLQAASSQLQVEQNKVTTVTEKLIEETKRALKSKTDVEEKMYSVTKERDDLKNKLKAEEEKGNDLLSRVNMLKNRLQSLEAIEKDFLKNKLNQDSGKSTTALHQENNKIKELSQEVERLKLKLKDMKAIEDDLMKTEDEYETLERRYANERDKAQFLSKELEHVKMELAKYKLAEKTETSHEQWLFKRLQEEEAKSGHLSREVDALKEKIHEYMATEDLICHLQGDHSVLQKKTKSTRKQEQRFRKRD

SEQ ID NO: 15 - protein, 1135 aa, Homo sapiens
MRSRGSDTEGSAQKKFPRHTKGHSFQGPKNMKHRQQDKDSPSESDVILPCPKAEKPHSGNGHQAEDLSRDDLLFLLSILEGELQARDEVIGILKAEKMDLALLEAQYGFVTPKKVLEALQRDAFQAKSTPWQEDIYEKPMNELDKVVEKHKESYRRILGQLLVAEKSRRQTILELEEEKRKHKEYMEKSDEFICLLEQECERLKKLIDQEIKSQEEKEQEKEKRVTTLKEELTKLKSFALMVVDEQQRLTAQLTLQRQKIQELTTNAKETHTKLALAEARVQEEEQKATRLEKELQTQTTKFHQDQDTIMAKLTNEDSQNRQLQQKLAALSRQIDELEETNRSLRKAEEELQDIKEKISKGEYGNAGIMAEVEELRKRVLDMEGKDEELIKMEEQCRDLNKRLERETLQSKDFKLEVEKLSKRIMALEKLEDAFNKSKQECYSLKCNLEKERMTTKQLSQELESLKVRIKELEAIESRLEKTEFTLKEDLTKLKTLTVMFVDERKTMSEKLKKTEDKLQAASSQLQVEQNKVTTVTEKLIEETKRALKSKTDVEEKMYSVTKERDDLKNKLKAEEEKGNDLLSRVNMLKNRLQSLEAIEKDFLKNKLNQDSGKSTTALHQENNKIKELSQEVERLKLKLKDMKAIEDDLMKTEDEYETLERRYANERDKAQFLSKELEHVKMELAKYKLAEKTETSHEQWLFKRLQEEEAKSGHLSREVDALKEKIHEYMATEDLICHLQGDHSVLQKKLNQQENRNRDLGREIENLTKELERYRHFSKSLRPSLNGRRISDPQVFSKEVQTEAVDNEPPDYKSLIPLERAVINGQLYEESENQDEDPNDEGSVLSFKCSQSTPCPVNRKLWIPWMKSKEGHLQNGKMQTKPNANFVQPGDLVLSHTPGQPLHIKVTPDHVQNTATLEITSPTTESPHSYTSTAVIPNCGTPKQRITILQNASITPVKSKTSTEDLMNLEQGMSPITMATFARAQTPESCGSLTPERTMSPIQVLAVTGSASSPEQGRSPEPTEISAKHAIFRVSPDRQSSWQFQRSNSNSSSVITTEDNKIHIHLGSPYMQAVASPVRPASPSAPLQDNRTQGLINGALNKTTNKVTSSITITPTATPLPRQSQITVEPLLLPH

SEQ ID NO: 16 - protein, 1133 aa, Homo sapiens [sequence omitted]

SEQ ID NO: 17 - protein, 1133 aa, Homo sapiens
MRSRGSDTEGSAQKKFPRHTKGHSFQGPKNMKHRQQDKDSPSESDVILPCPKAEKPHSGNGHQAEDLSRDDLLFLLSILEGELQARDEVIGILKAEKMDLALLEAQYGFVTPKKVLEALQRDAFQAKSTPWQEDIYEKPMNELDKVVEKHKESYRRILGQLLVAEKSRRQTILELEEEKRKHKEYMEKSDEFICLLEQECERLKKLIDQEIKSQEEKEQEKEKRVTTLKEELTKLKSFALMVVDEQQRLTAQLTLQRQKIQELTTNAKETHTKLALAEARVQEEEQKATRLEKELQTQTTKFHQDQDTIMAKLTNEDSQNRQLQQKLAALSRQIDELEETNRSLRKAEEELQDIKEKISKGEYGNAGIMAEVEELRKRVLDMEGKDEELIKMEEQCRDLNKRLERETLQSKDFKLEVEKLSKRIMALEKLEDAFNKSKQECYSLKCNLEKERMTTKQLSQELESLKVRIKELEAIESRLEKTEFTLKEDLTKLKTLTVMFVDERKTMSEKLKKTEDKLQAASSQLQVEQNKVTTVTEKLIEETKRALKSKTDVEEKMYSVTKERDDLKNKLKAEEEKGNDLLSRVNMLKNRLQSLEAIEKDFLKNKLNQDSGKSTTALHQENNKIKELSQEVERLKLKLKDMKAIEDDLMKTEDEYETLERRYANERDKAQFLSKELEHVKMELAKYKLAEKTETSHEQWLFKRLQEEEAKSGHLSREVDALKEKIHEYMATEDLICHLQGDHSVLQKKLNQQENRNRDLGREIENLTKELERYRHFSKSLRPSLNGRRISDPQVFSKEVQTEAVDNEPPDYKSLIPLERAVINGQLYEESENQDEDPNDEGSVLSFKCSQSTPCPVNRKLWIPWMKSKEGHLQNGKMQTKPNANFVQPGDLVLSHTPGQPLHIKVTPDHVQNTATLEITSPTTESPHSYTSTAVIPNCGTPKQRITILQNASITPVKSKTSTEDLMNLEQGMSPITMATFARAQTPESCGSLTPERTMSPIQVLAVTGSASSPEQGRSPEPTEISAKHAIFRVSPDRQSSWQFQRSNSNSSSVITTEDNKIHIHLGSPYMQAVASPVRPASPSAPLQDNRTQGLINGALNKTTNKVTSSITITPTATPLPRQSQITVSNIYN

SEQ ID NO: 18 - protein, 1135 aa, Homo sapiens [sequence omitted]

SEQ ID NO: 19 - protein, 197 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 20 - protein, 186 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 21 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 22 - protein, 160 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 23 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 24 - protein, 244 aa, Homo sapiens
GLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 25 - protein, 42 aa, Homo sapiens [sequence omitted]

(19) United States
(12) Patent Application Publication
(10) Pub. No.: US 2015/0093378 A1
(43) Pub. Date: Apr. 2, 2015

(54) Goodpasture Antigen Binding Protein Detection and Inhibition and its Use in Diabetes

(71) Applicant: FibroStatin, S.L., Valencia (ES)

(72) Inventors: Juan SAUS, Valencia (ES); Fernando REVERT, Valencia (ES); Francisco REVERT-ROS, Paterna (Valencia) (ES)

(21) Appl. No.: 14/497,924

(22) Filed: Sep. 26, 2014

Related U.S. Application Data
(60) Provisional application No. 61/883,792, filed on Sep. 27, 2013.

(51) Int. Cl. C07K 16/40; G01N 33/573; C07C 59/13

(52) U.S. Cl. CPC C07K 16/40; C07C 59/13; G01N 33/573; C07K 2317/76; G01N 2800/042; G01N 2333/912
USPC 424/133.1; 514/568; 435/7.4

(57) ABSTRACT

Disclosed herein are methods for treating type 2 diabetes, limiting development of type 2 diabetes, and treating a pre-diabetic state by administering to a subject in need thereof a GPBP inhibitor. Also disclosed herein are methods for diagnosing a pre-diabetic state and for diagnosing a propensity to develop type 2 diabetes by determining an amount of GPBP in a sample from a subject and comparing to control.

CROSS REFERENCE

This application claims priority to U.S. Provisional Patent Application Ser. No. 61/883,792 filed Sep. 27, 2013, incorporated by reference herein in its entirety.

### BACKGROUND OF THE INVENTION

The conformation of the non-collagenous (NC1) domain of the a3 chain of the basement membrane collagen IV (α3NC1) depends in part on phosphorylation. Goodpasture Antigen Binding Protein (GPBP) (WO 00/50607; WO 02/061430) is a novel non-conventional protein kinase that catalyzes the conformational isomerization of the α3NC1 domain during its supramolecular assembly, resulting in the production and stabilization of multiple α3NC1 conformers in basement membranes. Elevated levels of GPBP have been associated with the production of non-tolerized α3NC1 conformers, which conduct the autoimmune response mediating Goodpasture (GP) disease. In GP patients, autoantibodies against the α3NC1 (also known as GP antigen) cause a rapidly progressive glomerulonephritis and often lung hemorrhage, the two cardinal clinical manifestations of the GP disease.

COL4A3BP is a gene for regulation of structural protein supramolecular organization inside (i.e. myosin) and outside (i.e. collagen IV) of the cell. The primary gene product GPBP (also known as GPBP-1 or 77 kD GPBP) resides mainly in the extracellular compartment where it can be found circulating or bound to collagen IV. Interestingly, the gene also expresses alternative isoforms which remain mainly intracellular and soluble in the cytosol (i.e. GPBP-2 also known as GPBPΔ26 or CERT) or insoluble and associated to cellular membranes and organelles (i.e. GPBP-3 also known as 91 kD GPBP) (WO 00/50607; WO 2010/009856; Revert-Ros et al., 2011, J Biol. Chem 286, 35030-35043).

### SUMMARY OF THE INVENTION

In one aspect, the present invention provides methods for treating type 2 diabetes (T2D) or limiting development of T2D, comprising administering to a subject in need thereof an effective amount of a GPBP inhibitor to treat T2D or limit development of T2D.

In another aspect, the present invention provides methods for treating a pre-diabetic state, comprising administering to a subject in need thereof an effective amount a GPBP inhibitor, including but not limited to an anti-GPBP antibody, to treat the pre-diabetic state.

In a further aspect, the present invention provides methods for diagnosing a pre-diabetic state comprising

(a) determining an amount of GPBP in a sample from a subject at risk of being in a pre-diabetic state; and

(b) comparing the amount of GPBP in the sample to an amount of GPBP in a control sample;

wherein an increase in the amount of GPBP in the sample compared to the control sample indicates the presence of a pre-diabetic state in the subject.

In another aspect, the present invention provides methods for diagnosing a propensity to develop T2D, comprising

(a) determining an amount of GPBP in a sample from a subject at risk of having a pre-diabetic state; and

(b) comparing the amount of GPBP in the sample to an amount of GPBP in a control sample;

wherein an increase in the amount of GPBP in the sample compared to the control sample indicates a propensity to develop T2D in the subject.

### DESCRIPTION OF THE FIGURES

FIG. 1. Elevated extracellular glucose levels induces GPBP expression. C2C12 cells were differentiated for 5 days in DMEM (Dulbecco's modified Eagle's medium) supplemented with 2% horse serum and antibiotics. Cultures were washed with PBS (phosphate buffered saline) and incubated for another 24-hour period with DMEM supplemented with 2% horse serum containing glucose at the indicated concentrations. Cells were washed with PBS and lysed in 50 mM Tris-HCl pH 7.0, 150 mM NaCl, 1% Triton X-100, 1 mM PMSF (phenyl-methylsulfonyl fluoride), 10 mg/mL leupeptin for 30 min at 4° C. Lysates were cleared by centrifugation (16 000×g, 5 min, 4° C.), the total protein concentration of the supernatants was determined (Bio-Rad) and similar quantities (50 μg) of each extract were analyzed by Western blot with anti-GPBP monoclonal antibodies (mAb) N27 and anti-tubulin (Sigma), as elsewhere indicated.

FIG. 2. GPBP kinase inhibition enhances glucose consumption under increased extracellular glucose concentrations. A, A549 cells were cultured in DMEM plus 10% fetal bovine serum and penicillin/streptomycin with the indicated concentrations of glucose during 24 h. Then cells were lysed in 25 mM Tris-HCl pH 7.5, 150 mM NaCl, 1% Triton X-100, 0.1% SDS, 1 mM PMSF, 10 μg/mL leupeptin, 20 mM NaF at 4° C. during 30 min. Lysates were cleared by centrifugation at 16,000×g for 5 min (4° C.) and the protein concentration of supernatants was determined (Bio-Rad). Similar amounts (50 μg) of each lysate were analyzed by Western blot for detection of AS160 protein phosphorylated at Ser 318 (P Ser 318 AS160), GPBP and tubulin, using primary antibodies obtained from Cell Signaling Technology, Fibrostatin SL and Sigma, respectively, and HRP-conjugated anti-rabbit IgG and anti-mouse IgG secondary antibodies from Promega. Development was performed with Amersham ECL Prime Western Blotting Detection Reagent (GE Healthcare Lifesciences) and image acquisition with an ImageQuant LAS 4000 Mini device (GE Healthcare Lifesciences). B, A549 cells were cultured in DMEM: F-12 (315 mg glucose/dL) supplemented with 10% fetal bovine serum and penicillin/streptomycin with the indicated concentrations of T12 during 14 h. After incubations cells were lysed as in A and protein concentrations in supernatants were similarly assessed. Glucose concentrations in the media were determined at the beginning and at the end of the incubation period with a Glucocard G+ meter (Arkray Factory, Inc.), and glucose uptake by cells was calculated using the protein concentrations of the corresponding lysates for normalization purposes. Similar results were obtained with doxorubicin (0.5 μM) (data not shown), a different GPBP kinase inhibitor (WO/2014/006020). C, A549 cells were incubated in DMEM: F-12 (315 mg glucose/dL) plus 10% fetal bovine serum and penicillin/streptomycin during 20 h in absence (Control) or presence of T12 (50 μM), and lysed as in A. Similar amounts (50 μg) of each lysate were analyzed by Western blot for detection of AS160 protein phosphorylated at Ser 318 (P Ser 318 AS160) or of tubulin as in A.

FIG. 3. GPBP kinase inhibition also enhances glucose consumption under physiological extracellular glucose concentrations. C2C12 mouse myoblasts were differentiated to myotubes in DMEM supplemented with 2% horse serum and penicillin/streptomycin during 10 days, and then incubated in DMEM plus 0.25% horse serum with an initial glucose concentration of 115 mg/dL, in absence or presence of T12 (50 μM) during the indicated times. Glucose concentrations in media were determined with a Glucocard™ G+ meter (Arkray Factory, Inc.). Shown are means±SEM (n=2). Differences between the indicated groups were statistically significant according to t-Student test analysis. *P=0.0152; ***P=0.0005.

FIG. 4. Irs2^{-/-} mice displayed increased collagen IV expression. Kidneys were collected from male wild type (WT, n=9), normoglycemic Irs2^{-/-} (Irs2^{-/-}, n=9) and diabetic Irs2^{-/-} (n=9) mice at 8-12 weeks of age and embedded in paraffin for immunohistochemical analysis of sections with anti-type IV collagen antibodies from EMD Millipore. Mice were considered diabetic when serum glucose levels under fasting were greater than 120 mg/dL. In the composite the level of collagen IV expression is represented in a black (no expression) and white (expression) color range. Shown are representative images. Original magnification: ×400.

FIG. 5. Irs2^{-/-} mice displayed increased COL4A3BP expression. Kidneys were collected from male wild type (WT, n=9), normoglycemic Irs2^{-/-} (Irs2^{-/-}, n=9) and diabetic Irs2^{-/-} (n=9) mice at 8-12 weeks of age and embedded in paraffin for immunohistochemical analysis of sections with anti-GPBP mAb N26 from Fibrostatin SL. Mice were considered diabetic when serum glucose levels under fasting were greater than 120 mg/dL. In the composite the level of GPBP expression is represented in a black (no expression) and white (expression) color range. Shown are representative images. Original magnification: ×400.

FIG. 6. Circulating GPBP levels are augmented in normoglycemic Irs2-deficient mice. Blood samples were collected from wild type (WT, n=12), normoglycemic Irs2^{-/-} (Irs2^{-/-}, n=12) and diabetic Irs2^{-/-} (n=19) mice at the time of sacrifice (8-12 weeks of age) and circulating GPBP levels were measured using mAb N26 as capturing and mAb N27 as detecting antibodies in a prototype sandwich ELISA essentially as described in WO 2010/009856. Mice were considered diabetic when serum glucose levels under fasting were greater than 120 mg/dL. *P<0.05, ***P<0.001.

FIG. 7. Effect of anti-GPBP treatment on blood glucose in 10-week old mice. Different groups of mice received weekly intraperitoneal injections of anti-GPBP mAb N26 or mouse IgG (Sigma) at 1 μg/g of body weight, and fasting blood glucose (tail vein) was monitored weekly using a glucometer. Males. Wild type treated (WT+mAb N26, n=2), wild type control (WT+Cont IgG, n=2), Irs2^{-/-} treated (KO+mAb 26, n=2), Irs2^{-/-} control (KO+Cont IgG, n=2). Females. Wild type treated (n=2), wild type control (n=2), Irs2^{-/-} treated (n=3), irs2^{-/-} control (n=2).

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

Unless clearly indicated otherwise by the context, embodiments disclosed for one aspect of the invention can be used in other aspects of the invention as well, and in combination with embodiments disclosed in other aspects of the invention.

In one aspect, the present invention provides methods for treating T2D, comprising administering to a subject in need thereof an amount effective of a GPBP inhibitor to treat T2D. In this aspect, the subject is one who has been diagnosed with T2D. In another aspect, the invention provides methods for limiting development of T2D, comprising administering to a subject in need thereof an amount effective of a GPBP inhibitor to limit development of T2D. In this aspect, the subject can be any subject that is at risk of developing T2D, including but not limited to those subjects with one or more risk factors selected including, but not limited to obesity, a sedentary lifestyle, poor eating habits (ex: too much fat, not enough fiber, too many simple carbohydrates, etc.), family history of pre-diabetic state and/or T2D, age 50 or older, high blood pressure, high cholesterol, and history of gestational diabetes.

In a further aspect, the invention provides methods for treating a pre-diabetic state, comprising administering to a subject in need thereof an amount effective of a GPBP inhibitor to treat the pre-diabetic state. In this aspect, the subject is one who has a pre-diabetic state. As used herein, a “pre-diabetic state” is the state in which some but not all of the diagnostic criteria for diabetes are met. Thus, the pre-diabetic state may comprise: 1) having impaired fasting glucose tolerance, which is a condition whereby the response of beta cells to an oral glucose challenge (OGT) is deficient or 2) having consistently elevated fasting glucose (IFG), which is a condition in which the fasting blood glucose is elevated above what is considered normal levels but is not high enough to be classified as diabetes mellitus. The pre-diabetic state may be associated with insulin resistance and increased risk of cardiovascular pathology. Individuals with a pre-diabetic state are at a relatively high risk of developing T2D.

As used herein, “GPBP” will refer to any GPBP isoform, i.e.: GPBP-1 (SEQ ID NO:10), GPBP-2 (SEQ ID NO:11), and GPBP-3 (SEQ ID NO:12). GPBP-1 (also known as 77 kD GPBP) resides mainly in the extracellular compartment where it can be found circulating or bound to collagen IV. GPBP-2 (also known as GPBPΔ26 or CERT) is a GPBP-1 variant of 598-residue-long generated by mRNA alternative exon splicing that remains mainly intracellular and cytosolic. GPBP-3 (also known as 91 kD GPBP) is a GPBP-1 variant of 707-residue-long arising from alternative non-canonical mRNA translation initiation that remains mainly intracellular and associated with cellular membranes including the outer side of the plasma membrane and organelles.

As used herein a “GPBP inhibitor” will refer to any compound or molecule that reduces the activity or the expression of all or individual GPBP isoforms.

As demonstrated by the inventors in the examples that follow, GPBP-1 mediates pathogenesis in an animal model of T2D, and inhibition of GPBP-1 activity delayed or precluded hyperglycemia. Thus, inhibitors of GPBP-1 expression or activity can be used to treat or limit development of T2D, or to treat those with a diabetic or pre-diabetic state. The inventors have recently discovered (data not shown) that GPBP-1 performs at least three key activities related through a cycle: protein kinase (cytoplasm and, eventually, the nucleus and the endoplasmic reticulum); chaperone (endoplasmic reticulum) and inflammatory “cytokine” (extracellular compartment). The cycle's progression toward the cell exterior is regulated in part by GPBP-3 expression (WO 2010/009856) and is completed because the extracellular GPBP-1 is recaptured and returns to the cytoplasm. The evidence also shown that GPBP-2 expression can compensate GPBP-1 deficiency in vivo (Revert-Ros et al., 2011, J Biol. Chem 286, 35030-35043) supporting that GPBP-2 expression is somehow contributing to the normal progression of the GPBP cycle. The existence of the GPBP cycle with intra- and extracellular stages means that a disorder caused by exaggerated activity in the cytosol can be directly interfered with by an inhibitor compound, but also indirectly by either an inhibitor of the activity in the ER or an antibody that blocks extracellular activity; and vice versa, a disorder caused by exaggerated extracellular activity can be directly interfered with by a blocking antibody and indirectly by an inhibitor of intracellular activity. Thus, in light of the GPBP cycle inhibitors of any isoform of GPBP (GPBP-1, GPBP-2, and/or GPBP-3) can be used to treat or limit development of T2D, or to treat those with a diabetic or pre-diabetic state.

In all aspects and embodiments, the subject may be any mammal, including but not limited to a human, dog, cat, horse, or livestock (cow, sheep, etc.). In a most preferred embodiment, the subject is a human subject.

As used herein, “treat” or “treating” means accomplishing one or more of the following: (a) reducing the severity of the disorder; (b) limiting or preventing development of symptoms or complications characteristic of the disorder(s) being treated; (c) inhibiting worsening of symptoms or complications characteristic of the disorder(s) being treated; (d) limiting or preventing recurrence of symptoms or complications in patients that were previously symptomatic for the disorder(s).

Symptoms and complications of T2D that can be limited with the methods of the invention include, but are not limited to hyperglycemia, insulin resistance, diabetic nephropathy, diabetic neuropathy, diabetic retinopathy, proteinuria, impaired glomerular clearance, diabetic circulatory disorders, and kidney failure. Any amount of limiting of these symptoms/complications is of great benefit to a subject with T2D.

The methods of the invention can be used to treat a subject with pre-diabetes by, for example, limiting/slowing progression of hyperglycemia, insulin resistance, and/or cardiovascular pathologies in the subject and/or limiting/slowing progression of the subject to T2D.

In one embodiment, the subject has increased circulating GPBP relative to control. This embodiment may help identify subjects that may most benefit from treatment. Such “increase” can be any amount of increase in GPBP relative to control (such as control sample from normal subject, or previously determined “normal” levels of GPBP in a control population), for example, 5%, 10%, 15%, 20%, 25%, 50%, 75%, 100%, or greater. In one embodiment, a normal value of GPBP as a reference for a standard curve is less than 10 ng/ml in plasma. In various embodiments, a normal GPBP range may be between 1 ng/ml-10 ng/ml in plasma. Thus, in one embodiment, subjects identified as having more than 10 ng/ml of GPBP in their plasma are treated according to the methods of the invention. Methods for determining the amount of circulating GPBP are known (see WO 2010/009856 and U.S. Pat. No. 7,935,492).

Any suitable GPBP inhibitor may be used in the methods of the invention. In one embodiment, the GPBP inhibitor comprises an anti-GPBP antibody, such as a monoclonal or polyclonal antibody. As used herein, “anti-GPBP antibody” means that the antibodies bind to all or individual GPBP isoforms. In a preferred embodiment that can be combined with any other embodiment, the antibody is a monoclonal antibody, such as a humanized monoclonal antibody. The term antibody as used herein is intended to include antibody fragments thereof which are selectively reactive with the polypeptides of the invention, or fragments thereof. Antibodies can be fragmented using conventional techniques or synthesized through genetic engineering using recombinant DNA, and the fragments screened for utility in the same manner as described above for whole antibodies. For example, F(ab′)2 fragments can be generated by treating antibody with pepsin. The resulting F(ab′)2 fragment can be treated with papain to produce Fab fragments. Examples of monoclonal antibody fragments include (i) a Fab fragment, a monovalent fragment consisting essentially of the VL, VH, CL and CH1 domains; (ii) F(ab)2 and F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, such as scFV, tandem di-scFV, diabodies, tri(a)bodies, etc. (v) a dAb fragment (Ward et al., (1989) Nature 341:544-546), which consists essentially of a VH domain; and (vi) one or more isolated CDRs or a functional paratope.

In various further embodiments, the GPBP inhibitor comprises one or more inhibitors selected from the group consisting of:

(a) an aptamer selective for GPBP;

(c) an inhibitory nucleic acid selective for GPBP mRNA;

(d) a peptide comprising an amino acid sequence of general formula X1-SHCIX2-X3 (SEQ ID NO: 1)
wherein X1 is 0-10 amino acids of the sequence ATTAGILATL (SEQ ID NO: 2);
X2 is E or Q; and
X3 is 0-10 amino acids of the sequence LMVKREDSWQ (SEQ ID NO: 3);

(e) a peptide comprising an amino acid sequence selected from the group consisting of SHCIE (SEQ ID NO: 4), SHCIQ (SEQ ID NO: 5), ILATLSHCIELMVKR (SEQ ID NO: 6), ILATLSHCIQLMVKR (SEQ ID NO: 7), and LATLSHCIELMVKR (SEQ ID NO: 8); and

(f) I-20:

(SEQ ID NO: 9)
RDEVIGILKAEKMDLALLEAQYGFVTPKKVLEALQRDAFQAKSTPWQEDI
YEKPMNELDKVVEKHKESYRRILGQLLVAEKSRRQTILELEEEKRKHKEY
MEKSDEFICLLEQECERLKKLIDQEIKSQEEKEQEKEKRVTTLKEELTKL
KSFALMVVDEQQRLTAQLTLQRQKIQELTTNAKETHTKLALAEARVQEEE
QKATRLEKELQTQTTKFHQDQDTIMAKLTNEDSQNRQLQQKLAALSRQID
ELEETNRSLRKAEEE.

Sequences of the polypeptides recited in the above are known in the art; see, for example, U.S. Pat. Nos. 7,326,768; 7,935,492; 6,579,969; and 7,147,855.

Synthesis of the other GPBP inhibitors disclosed herein can be carried out by those of skill in the art, based on the teachings in WO2011/054530.

The peptides can further be derivatized to provide enhanced half-life, such as by the addition of polyethylene glycol (PEG) or as otherwise known in the art. The peptides may comprise L-amino acids, D-amino acids (which are resistant to L-amino acid-specific proteases in vivo), a combination of D- and L-amino acids, and various “designer” amino acids (e.g., β-methyl amino acids, Cα-methyl amino acids, and Nα-methyl amino acids, etc.) to convey special properties. Synthetic amino acids include ornithine for lysine, and norleucine for leucine or isoleucine.

In addition, the peptides can have peptidomimetic bonds, such as ester bonds, to prepare peptides with novel properties. For example, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a peptide would be resistant to protease activity, and would possess an extended half-live in vivo.

The term “peptide” is used in its broadest sense to refer to a sequence of subunit amino acids, amino acid analogs, or peptidomimetics. The subunits are linked by peptide bonds, although the peptide can comprise further moieties that are not necessarily linked to the peptide by a peptide bond. For example, as discussed above, the peptide can further comprise a non-amino acid molecule that contains an aromatic ring.

The peptides described herein may be chemically synthesized or recombinantly expressed. Recombinant expression can be accomplished using standard methods in the art, as disclosed above. Such expression vectors can comprise bacterial or viral expression vectors, and such host cells can be prokaryotic or eukaryotic.

The peptide/antibody inhibitors may be administered together in a pharmaceutical composition with a pharmaceutically acceptable carrier. The pharmaceutical composition may comprise in addition to the peptide/antibody inhibitor (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

In another embodiment, the GPBP inhibitor is a compound of formula (A) as described in WO2011/054530:

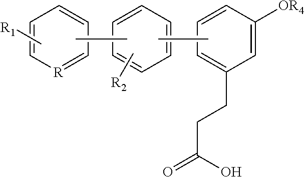

or a pharmaceutically acceptable salt thereof, wherein:

R is selected from N and CR₃; R₁ is hydrogen or C₁-C₆ alkoxy; R₂ is C₁-C₆ alkyl or halo(C₁-C₆ alkyl); R₃, if present, is C₁-C₆ alkyl or halo(C₁-C₆ alkyl); and R₄ is H or C₁-C₆ alkyl.

In another preferred embodiment of the compound of formula (A), R₁ is hydrogen or methoxy; R₂ is methyl, fluoromethyl, or difluoromethyl; R₃, if present, is methyl or trifluoromethyl; and R₄ is H or methyl.

In various further preferred embodiments, R₁ is hydrogen and/or R₄ is methyl. In further preferred embodiments, the compound of formula (A) is selected from the group consisting of:

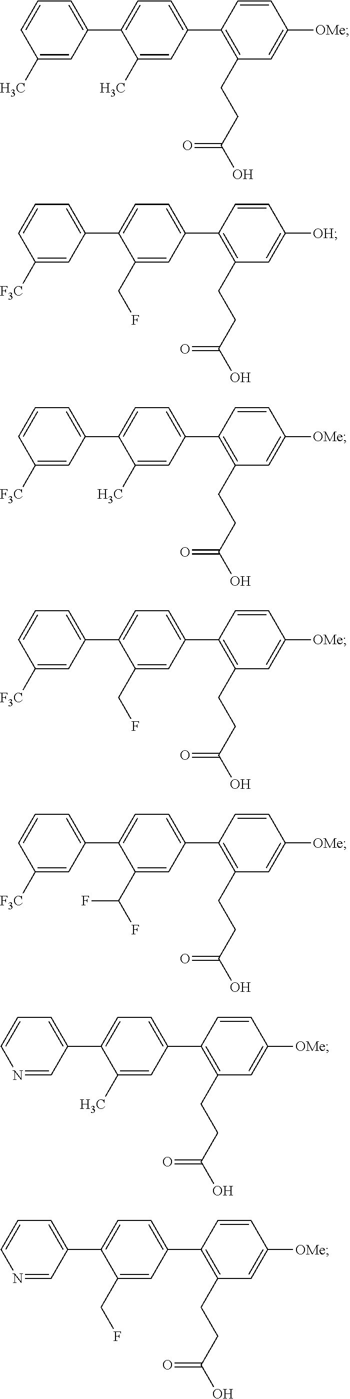

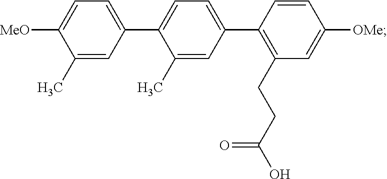 [SMILES: COc1ccc(-c2ccc(-c3ccc(OC)c(C)c3)c(C)c2)c(CCC(=O)O)c1]

or a pharmaceutically acceptable salt thereof. In a most preferred embodiment, the compound is

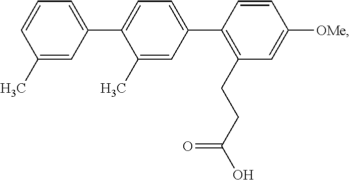 [SMILES: COc1ccc(-c2ccc(-c3cccc(C)c3)c(C)c2)c(CCC(=O)O)c1]

or a pharmaceutically acceptable salt thereof.

In another preferred embodiment, the GPBP inhibitor is a compound of formula (B):

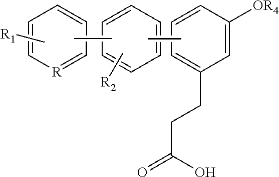

or a pharmaceutically acceptable salt thereof, wherein:
R is selected from N and CR₃;
   R₃ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), or hydroxy(C₁-C₆ alkyl); and
R₄ is H, C₁-C₆ alkyl, —C(O)(C₁-C₂₀ alkyl), or —(CH₂)₁₋₅—C(O)OH.

In another embodiment the GPBP inhibitor of formula B is a compound of formula (C):

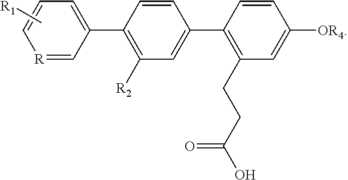

or a pharmaceutically acceptable salt thereof.

In one embodiment of the GPBP inhibitors of formulae (B) or (C), R is selected from N and CR₃; R₃ is selected from the group consisting of hydrogen, halogen, hydroxy, C₁-C₆ alkyl, C₂-C₆ alkenyl, C₂-C₆ alkynyl, halo(C₁-C₆ alkyl), C₁-C₆ alkoxy, or halo(C₁-C₆ alkoxy; R₁ is hydrogen, hydroxy, C₁-C₆ alkyl, or C₁-C₆ alkoxy; R₂ is C₁-C₆ alkyl, halo(C₁-C₆ alkyl), or hydroxy(C₁-C₆ alkyl); and R₄ is H, C₁-C₆ alkyl, —C(O)(C₁-C₂₀ alkyl), or —(CH₂)₁₋₅—C(O)OH.

In another embodiment the GPBP inhibitor is a compound having the formula:

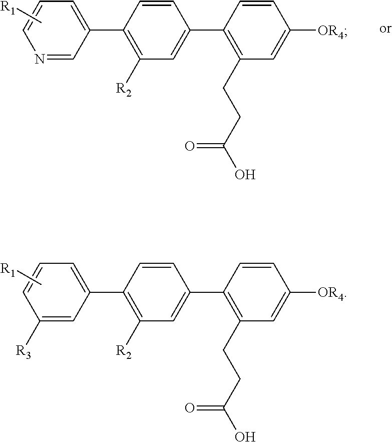

In various embodiments of any embodiment of the compounds of formulae (B) and (C), R₂ may be C₁-C₆ alkyl, halo(C₁-C₆ alkyl), or hydroxy(C₁-C₆ alkyl). For example, R₂ may be C₁-C₆ alkyl, methyl, halo(C₁-C₆ alkyl), fluoromethyl, difluoromethyl, hydroxymethyl, or hydroxy(C₁-C₆ alkyl).

In various further embodiments of any embodiment of the compounds of formulae (B) and (C), R₄ may be H, C₁-C₆ alkyl, methyl, propyl, —C(O)(C₁-C₂₀ alkyl), or —(CH₂)₁₋₅—C(O)OH.

In various further embodiments of any embodiment of the compounds of formulae (B) and (C), R₁ may be hydrogen or methoxy.

In various further embodiments of any embodiment of the compounds of formulae (B) and (C), R₃ may be C₁-C₆ alkyl, halo(C₁-C₆ alkyl), C₁-C₆ alkoxy, halo(C₁-C₆ alkoxy), methyl, or trifluoromethyl.

In a further embodiment of the compounds of formulae (B) and (C), R₁ is hydrogen or C₁-C₆ alkoxy; R₂ is hydroxy(C₁-C₆ alkyl); R₃, if present, is C₁-C₆ alkyl or halo(C₁-C₆ alkyl); and R₄ is H or C₁-C₆ alkyl.

In another embodiment of the compounds of formulae (B) and (C), R₁ is hydrogen or methoxy; R₂ is hydroxymethyl; R₃, if present, is methyl or trifluoromethyl; and R₄ is H or methyl.

In another embodiment the GPBP inhibitor is a compound of formula (D):

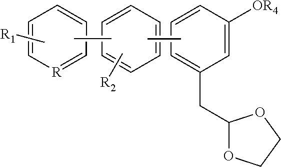

or a pharmaceutically acceptable salt thereof, wherein:
R is selected from N and CR₃;
   R₃ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, or —CH═CH—C(O)(C₁-C₆ alkoxy); and
R₄ is hydrogen, C₁-C₆ alkyl, or (aryl)C₁-C₆ alkyl.

In one embodiment, the compound of formula (D) is of the formula:

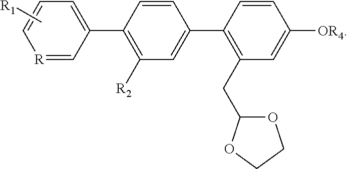

In one embodiment of any embodiment of the compound of formula (D), R₁ is hydrogen. In another embodiment of any embodiment of the compound of formula (D) R is CR₃, and R₃ is formyl(C₁-C₆ alkyl). In another embodiment, R₃ is —COH. In various embodiments, R₂ may be —CH═CH—C(O)OH; —CH═CH—C(O)(C₁-C₆ alkoxy); or —CH═CH—C(O)(OEt). In various further embodiments, R₄ may be (aryl)C₁-C₆ alkyl; or CH₂-Ph. In a preferred embodiment, the compound of formula D is a compound is of the formula:

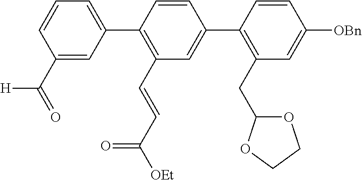 [SMILES: CCOC(=O)/C=C/c1cc(-c2ccc(OCc3ccccc3)cc2CC2OCCO2)ccc1-c1cccc(C=O)c1]

, or a pharmaceutically acceptable salt thereof.

In another embodiment, the GPBP inhibitors comprise compounds of formula (I):

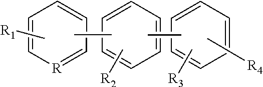

or a pharmaceutically acceptable salt thereof, wherein:
R is selected from N and CR₅;
   R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, (aryl)C₁-C₆ alkyl, or (heteroaryl)C₁-C₆ alkyl;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH2, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), (aryl)C₁-C₆ alkyl, or (heteroaryl)C₁-C₆ alkyl; and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH2, —(CH₂)₁₋₅—C(O)NH(C₁-C₆ alkyl), —(CH₂)₁-₅—C(O)N(C₁-C₆ alkyl)₂, —CH═CH—C(O)OH, —CH═CH—C(O)(C₁-C₆ alkoxy), —O(CH₂)₁₋₅—C(O)OH, —O(CH₂)₁₋₅—C(O)(C₁-C₆ alkoxy), (aryl)C₁-C₆ alkyl, or (heteroaryl)C₁-C₆ alkyl.

In another embodiment, the GPBP inhibitors are compounds of formula (II):

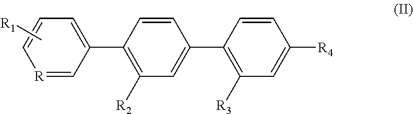

In another embodiment, the GPBP inhibitors are compounds of formulae (I) or (II) wherein:

R is selected from N and CR₅;

R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), and —(CH₂)₁₋₅—C(O)NH₂;

R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);

R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), or —(CH₂)₁₋₅—C(O)NH₂;

R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁-₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, or —CH═CH—C(O)(C₁-C₆ alkoxy); and

R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁-₅—C(O)N(C₁-C₆ alkyl)₂, —CH═CH—C(O)OH, —CH═CH—C(O)(C₁-C₆ alkoxy), —O(CH₂)₁₋₅—C(O)OH, or —O(CH₂)₁₋₅—C(O)(C₁-C₆ alkoxy).

In another embodiment, the GPBP inhibitors are compounds of formulae (I) or (II) wherein:

R is selected from N and CR₅;

R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);

R₁ is hydrogen, halogen, hydroxy, C₁-C₆ alkyl, halo(C₁-C₆ alkyl), C₁-C₆ alkoxy, or halo(C₁-C₆ alkoxy);

R₂ is C₁-C₆ alkyl, halo(C₁-C₆ alkyl), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), or (C₁-C₆ alkyl)sulfanyl(C₁-C₆ alkyl);

R₃ is C₁-C₆ alkyl, —(CH₂)₁₋₅—C(O)OH, —(CH₂)₁₋₅—C(O)(C₁-C₆ alkoxy), —(CH₂)₁₋₅—C(O)NH2, —(CH₂)₁₋₅—C(O)NH(C₁-C₆ alkyl), —(CH₂)₁-₅—C(O)N(C₁-C₆ alkyl)₂, —CH═CH—C(O)OH, or —CH═CH—C(O)(C₁-C₆ alkoxy); and

R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH2, —(CH₂)₁₋₅—C(O)NH(C₁-C₆ alkyl), —(CH₂)₁-₅—C(O)N(C₁-C₆ alkyl)₂, —CH═CH—C(O)OH, —CH═CH—C(O)(C₁-C₆ alkoxy), —O(CH₂)₁₋₅—C(O)OH, or —O(CH₂)₁₋₅—C(O)(C₁-C₆ alkoxy).

In another embodiment, the GPBP inhibitors are compounds of formula (II), wherein R is N. These compounds can be represented by formula (III):

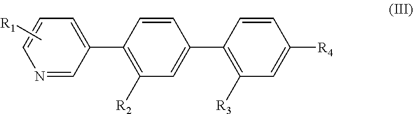

In yet another embodiment, the GPBP inhibitors are compounds of formula (II), wherein R is CR₅. These compounds can be represented by formula (IV):

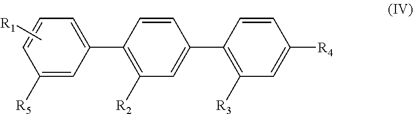

In one embodiment, the GPBP inhibitors are compounds as described above with reference to any of formulae (I)-(IV), wherein R₁ is hydrogen, hydroxy, or C₁-C₆ alkoxy. In one embodiment, the GPBP inhibitors are compounds as described above with reference to any of formulae (I)-(IV), wherein R₁ is hydrogen. In another embodiment, the GPBP inhibitors are compounds as described above with reference to any of formulae (I)-(IV), wherein R₂ is C₁-C₆ alkyl, halo(C₁-C₆ alkyl), hydroxy(C₁-C₆ alkyl), formyl(C₀-C₆ alkyl), amino(C₁-C₆ alkyl), or sulfanyl(C₁-C₆ alkyl). In yet another embodiment, the GPBP inhibitors are compounds as described above with reference to any of formulae (I)-(IV), wherein R₂ is C₁-C₆ alkyl, halo(C₁-C₆ alkyl), hydroxy(C₁-C₆ alkyl), or formyl(C₀-C₆ alkyl).

In yet another embodiment, the GPBP inhibitors are compounds as described above with reference to any of formulae (I)-(IV), wherein R₂ can be C₁-C₆ alkyl, halo(C₁-C₆ alkyl), or hydroxy(C₁-C₆ alkyl). For example, in certain embodiments R₂ can be C₁-C₆ alkyl such as methyl, ethyl, or isopropyl. In other embodiments, R₂ can be halo(C₁-C₆ alkyl) such as fluoromethyl, difluoromethyl, or trifluoromethyl. R₂ can, in certain embodiments, be hydroxy(C₁-C₆ alkyl). For example, the hydroxy(C₁-C₆ alkyl) can be hydroxymethyl, 1-hydroxyethyl, or 2-hydroxyethyl.

In another embodiment, the GPBP inhibitors are compounds as described above with reference to any of formulae (I)-(IV), wherein R₂ is C₁-C₆ alkyl. In certain embodiments R₂ is methyl. In another embodiment, the GPBP inhibitors are compounds as described above with reference to any of formulae (I)-(IV), wherein R₃ is C₁-C₆ alkyl, —(CH₂)₁₋₅—C(O)OH, —(CH₂)₁₋₅—C(O)(C₁-C₆ alkoxy), —CH═CH—C(O)OH, or —CH═CH—C(O)(C₁-C₆ alkoxy). In another embodiment, the GPBP inhibitors are compounds as described above with reference to any of formulae (I)-(IV), wherein R₃ is —(CH₂)₁₋₅—C(O)OH, —(CH₂)₁₋₅—C(O)(C₁-C₆ alkoxy), or —(CH₂)₁₋₅—C(O)NH₂.

In yet another embodiment, the GPBP inhibitors are compounds as described above with reference to any of formulae (I)-(IV), wherein R₃ is —(CH₂)₁₋₂—C(O)OH, or —(CH₂)₁₋₂—C(O)(C₁-C₆ alkoxy). For example, in certain embodiments R₃ can be —(CH₂)₂—C(O)OH, —(CH₂)₂—C(O)(OCH₃), —(CH₂)₂—C(O)(OCH₂CH₃), or —(CH₂)₂—C(O)(OC(CH₃)₃). In other embodiments, R₃ can be —(CH₂)₂—C(O)OH, or —(CH₂)₂—C(O)(OCH₂CH₃).

In another embodiment, the GPBP inhibitors are compounds as described above with reference to any of formulae (I)-(IV), wherein R₃ is —(CH₂)₁₋₂—C(O)OH. In one embodiment, R₃ is —(CH₂)₂—C(O)OH. In one embodiment, the GPBP inhibitors are compounds as described above with reference to any of formulae (I)-(IV), wherein R₄ is hydroxy, halogen, C₁-C₆ alkyl, C₁-C₆ alkoxy, halo(C₁-C₆ alkoxy), or benzyloxy. In another embodiment, the GPBP inhibitors are compounds as described above with reference to any of formulae (I)-(IV), wherein R₄ is hydroxy or C₁-C₆ alkoxy (e.g., methoxy). Preferably R₄ is C₁-C₆ alkoxy. In more preferred embodiment, R₄ is methoxy.

In one embodiment, the GPBP inhibitors are compounds as described above with reference to formulae (I), (II), or (IV), wherein R₅ is C₁-C₆ alkyl, halo(C₁-C₆ alkyl), C₁-C₆ alkoxy, or halo(C₁-C₆ alkoxy). In another embodiment, the GPBP inhibitors are compounds as described above with reference to formulae (I), (II), or (IV), wherein R₅ is C₁-C₆ alkyl, such as methyl. In yet another embodiment, the GPBP inhibitors are compounds as described above with reference to formulae (I), (II), or (IV), wherein R₅ is halo(C₁-C₆ alkyl), such as trifluoromethyl.

In certain embodiments, the GPBP inhibitors are compounds of any of formulae (I), (II), or (IV), wherein:

R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);

R₁ is hydrogen, halogen, hydroxy, C₁-C₆ alkyl, halo(C₁-C₆ alkyl), C₁-C₆ alkoxy, or halo(C₁-C₆ alkoxy);

R₂ is C₁-C₆ alkyl, halo(C₁-C₆ alkyl), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), or (C₁-C₆ alkyl)thio(C₁-C₆ alkyl);

R₃ is —(CH₂)₁₋₂—C(O)OH, —(CH₂)₁₋₂—C(O)(C₁-C₆ alkoxy), —(CH₂)₁₋₂—C(O)NH₂, —(CH₂)₁₋₂—C(O)NH(C₁-C₆ alkyl), —(CH₂)₁₋₂—C(O)N(C₁-C₆ alkyl)₂, —CH═CH—C(O)OH, —CH═CH—C(O)(C₁-C₆ alkoxy); and

R₄ is hydroxy, C₁-C₆ alkoxy, halo(C₁-C₆ alkoxy), or benzyloxy.

In certain embodiments, the GPBP inhibitors are compounds of any of formula (III), wherein:

R₁ is hydrogen, halogen, hydroxy, C₁-C₆ alkyl, halo(C₁-C₆ alkyl), C₁-C₆ alkoxy, or halo(C₁-C₆ alkoxy);

R₂ is C₁-C₆ alkyl, halo(C₁-C₆ alkyl), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), or (C₁-C₆ alkyl)thio(C₁-C₆ alkyl);

R₃ is —(CH₂)₁₋₂—C(O)OH, —(CH₂)₁₋₂—C(O)(C₁-C₆ alkoxy), —(CH₂)₁₋₂—C(O)NH₂, —(CH₂)₁₋₂—C(O)NH(C₁-C₆ alkyl), —(CH₂)₁₋₂—C(O)N(C₁-C₆ alkyl)₂, —CH═CH—C(O)OH, —CH═CH—C(O)(C₁-C₆ alkoxy); and

R₄ is hydroxy, C₁-C₆ alkoxy, halo(C₁-C₆ alkoxy), or benzyloxy.

In certain embodiments, the GPBP inhibitors are compounds of any of formulae (I), (II), or (IV), wherein R₁ is hydrogen; R₂ is C₁-C₆ alkyl, halo(C₁-C₆ alkyl), hydroxy(C₁-C₆ alkyl), or formyl(C₁-C₆ alkyl); R₃ is —(CH₂)₁₋₂—C(O)OH, —(CH₂)₁₋₂—C(O)(C₁-C₆ alkoxy), or —(CH₂)₁₋₂—C(O)NH₂; R₄ is hydroxy or C₁-C₆ alkoxy; and R₅ is C₁-C₆ alkyl, halo(C₁-C₆ alkyl), C₁-C₆ alkoxy, or halo(C₁-C₆ alkoxy).

In certain embodiments, the GPBP inhibitors are compounds of any of formula (III), wherein R₁ is hydrogen; R₂ is C₁-C₆ alkyl, halo(C₁-C₆ alkyl), hydroxy(C₁-C₆ alkyl), or formyl(C₁-C₆ alkyl); R₃ is —(CH₂)₁₋₂—C(O)OH, —(CH₂)₁₋₂—C(O)(C₁-C₆ alkoxy), or —(CH₂)₁₋₂—C(O)NH₂; R₄ is hydroxy or C₁-C₆ alkoxy; and R₅ is C₁-C₆ alkyl, halo(C₁-C₆ alkyl), C₁-C₆ alkoxy, or halo(C₁-C₆ alkoxy).

In certain embodiments, the GPBP inhibitors are compounds of any of formulae (I)-(IV), wherein:

R, if present, is selected from N and CR₅;

R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);

R₁ is hydrogen;

R₂ is C₁-C₆ alkyl;

R₃ is —(CH₂)₁₋₂—C(O)OH; and

R₄ is C₁-C₆ alkoxy.

In certain embodiments, the GPBP inhibitors are compounds of any of formulae (I)-(IV), wherein:

R, if present, is selected from N and CR₅;

R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);

R₁ is hydrogen;

R₂ is methyl;

R₃ is —(CH₂)₂—C(O)OH; and

R₄ is methoxy.

In one embodiment, the GPBP inhibitors are compounds of formula (V):

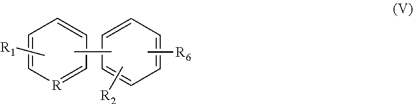

or a pharmaceutically acceptable salt thereof, wherein:

R is selected from N and CR₅;

R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, (aryl)C₁-C₆ alkyl, and (heteroaryl)C₁-C₆ alkyl;

R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);

R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH2, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), (aryl)C₁-C₆ alkyl, or (heteroaryl)C₁-C₆ alkyl; and

R₆ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH2, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —OS(O)₂CF₃, (aryl)C₁-C₆ alkyl, or (heteroaryl)C₁-C₆ alkyl.

In one embodiment, the compounds of formula (V) are of formula (VI):

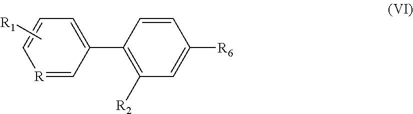

In another embodiment, the GPBP inhibitors are compounds of formulae (V) or (VI) wherein:

R is selected from N and CR₅;

R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), and —(CH₂)₁₋₅—C(O)NH₂;

R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);

R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH2, —CH═CH—C(O)OH, or —CH═CH—C(O)(C₁-C₆ alkoxy); and

R₆ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁-₅—C(O)N(C₁-C₆ alkyl)₂, —CH═CH—C(O)OH, —CH═CH—C(O)(C₁-C₆ alkoxy), or —OS(O)₂CF₃.

In another embodiment, the GPBP inhibitors are compounds of formulae (V) or (VI) wherein:

R is selected from N and CR₅;

R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);

R₁ is hydrogen, halogen, hydroxy, C₁-C₆ alkyl, halo(C₁-C₆ alkyl), C₁-C₆ alkoxy, or halo(C₁-C₆ alkoxy);

R₂ is C₁-C₆ alkyl, halo(C₁-C₆ alkyl), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —CH═CH—C(O)OH, or —CH═CH—C(O)(C₁-C₆ alkoxy); and

R₆ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), or —OS(O)₂CF₃.

In one embodiment, the GPBP inhibitors are compounds as described above with reference to any of formulae (V)-(VI), wherein R₁ is hydrogen.

In another embodiment, the GPBP inhibitors are compounds as described above with reference to any of formulae (V)-(VI), wherein R₂ is C₁-C₆ alkyl, halo(C₁-C₆ alkyl), hydroxy(C₁-C₆ alkyl), formyl(C₀-C₆ alkyl), amino(C₁-C₆ alkyl), sulfanyl(C₁-C₆ alkyl), —CH═CH—C(O)OH, or —CH═CH—C(O)(C₁-C₆ alkoxy).

In yet another embodiment, the GPBP inhibitors are compounds as described above with reference to any of formulae (V)-(VI), wherein R₂ is C₁-C₆ alkyl, halo(C₁-C₆ alkyl), hydroxy(C₁-C₆ alkyl), formyl(C₀-C₆ alkyl), —CH═CH—C(O)OH, or —CH═CH—C(O)(C₁-C₆ alkoxy).

In yet another embodiment, the GPBP inhibitors are compounds as described above with reference to any of formulae (V)-(VI), wherein R₂ can be C₁-C₆ alkyl, halo(C₁-C₆ alkyl), hydroxy(C₁-C₆ alkyl), or —CH═CH—C(O)(C₁-C₆ alkoxy). For example, in certain embodiments R₂ can be C₁-C₆ alkyl such as methyl, ethyl, or isopropyl. In other embodiments, R₂ can be halo(C₁-C₆ alkyl) such as fluoromethyl, difluoromethyl, or trifluoromethyl. R₂ can, in certain embodiments, be hydroxy(C₁-C₆ alkyl). For example, the hydroxy(C₁-C₆ alkyl) can be hydroxymethyl, 1-hydroxyethyl, or 2-hydroxyethyl.

In another embodiment, the GPBP inhibitors are compounds as described above with reference to any of formulae (V)-(VI), wherein R₂ is C₁-C₆ alkyl. In certain embodiments R₂ is methyl.

In one embodiment, the GPBP inhibitors are compounds as described above with reference to any of formulae (V)-(VI), wherein R₆ is hydroxy, halogen, C₁-C₆ alkyl, C₁-C₆ alkoxy, halo(C₁-C₆ alkoxy), benzyloxy, or —OS(O)₂CF₃.

In another embodiment, the GPBP inhibitors are compounds as described above with reference to any of formulae (V)-(VI), wherein R₆ is hydroxy or C₁-C₆ alkoxy (e.g., methoxy). In one embodiment, the GPBP inhibitors are compounds as described above with reference to formulae (V)-(VI), wherein R₅ is C₁-C₆ alkyl, halo(C₁-C₆ alkyl), C₁-C₆ alkoxy, or halo(C₁-C₆ alkoxy). In another embodiment, the GPBP inhibitors are compounds as described above with reference to formulae (V)-(VI), wherein R₅ is C₁-C₆ alkyl, such as methyl. In yet another embodiment, the GPBP inhibitors are compounds as described above with reference to formulae (V)-(VI), wherein R₅ is halo(C₁-C₆ alkyl), such as trifluoromethyl.

In certain embodiments, the GPBP inhibitors are compounds of any of formulae (V)-(VI), wherein:

R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, and amino(C₁-C₆ alkyl);

R₁ is hydrogen, halogen, hydroxy, C₁-C₆ alkyl, halo(C₁-C₆ alkyl), C₁-C₆ alkoxy, or halo(C₁-C₆ alkoxy);

R₂ is C₁-C₆ alkyl, halo(C₁-C₆ alkyl), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)thio(C₁-C₆ alkyl), or —CH═CH—C(O)(C₁-C₆ alkoxy); and

R₆ is hydroxy, C₁-C₆ alkoxy, halo(C₁-C₆ alkoxy), or —OS(O)₂CF₃.

In certain embodiments, the GPBP inhibitors are compounds of any of formulae (V)-(VI), wherein R₁ is hydrogen; R₂ is C₁-C₆ alkyl, halo(C₁-C₆ alkyl), hydroxy(C₁-C₆ alkyl), formyl(C₀-C₆ alkyl), or —CH═CH—C(O)(C₁-C₆ alkoxy); R₆ is hydroxy, C₁-C₆ alkoxy, or —OS(O)₂CF₃; and R₅ is C₁-C₆ alkyl, halo(C₁-C₆ alkyl), C₁-C₆ alkoxy, or halo(C₁-C₆ alkoxy).

Synthesis of the other GPBP inhibitors disclosed herein can be carried out by those of skill in the art, based on the teachings in WO2011/054530.

In one preferred embodiment, the GPBP inhibitor comprises or consists of 3-[4″-methoxy-3,2′-dimethyl-(1,1′;4′,1″)terphenyl-2″-yl]propionic acid; 3-[2′-methyl-4″-methoxy-3-(trifluoromethyl)-(1,1′;4′,1″)terphenyl-2″-yl]propionic acid; combinations thereof, or a pharmaceutically acceptable salt thereof.

The GBPB inhibitor compounds include pharmaceutically acceptable salts, esters, amides, and prodrugs thereof, including but not limited to carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” 1977. J. Pharm. Sci.,66: 1-19 which is incorporated herein by reference.)

Examples of pharmaceutically acceptable, non-toxic esters of the inhibitors include C₁-C₆ alkyl esters, wherein the alkyl group is straight or branched, substituted or unsubstituted, C₅-C₇ cycloalkyl esters, as well as arylalkyl esters such as benzyl and triphenylmethyl. C₁-C₄ alkyl esters are preferred, such as methyl, ethyl, 2,2,2-trichloroethyl, and tert-butyl. Esters of the compounds of the present invention may be prepared according to conventional methods.

Examples of pharmaceutically acceptable, non-toxic amides of the inhibitors include amides derived from ammonia, primary C₁-C₆ alkyl amines and secondary C₁-C₆ dialkyl amines, wherein the alkyl groups are straight or branched. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

The term “prodrug” refers to compounds that are rapidly transformed in vivo to yield the parent compound of the above formulae, for example, by hydrolysis in blood. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, —CH₂CH(CH₂CH₃)CH₂—.

The term “alkylene” refers to a bivalent alkyl group. An “alkylene chain” is a polymethylene group, i.e., —(CH₂)ₙ—, wherein n is a positive integer, preferably from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 2H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The term “halo” or “halogen” as used herein, means —Cl, —Br, —I or —F.

The terms “haloalkyl”, “haloalkenyl” and “haloalkoxy” refer to an alkyl, alkenyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The GPBP inhibitors are ordinarily combined with one or more adjuvants appropriate for the indicated route of administration. The inhibitors may be mixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the inhibitors may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The GPBP inhibitors can be administered as the sole active pharmaceutical agent, or they can be used in combination with one or more other compounds useful for carrying out the methods of the invention. When administered as a combination, the therapeutic agents can be formulated as separate compositions that are given at the same time or different times, or the therapeutic agents can be given as a single composition.

The GPBP inhibitors may be made up in a solid form (including granules, powders or suppositories) or in a liquid form (e.g., solutions, suspensions, or emulsions).

The GPBP inhibitors may be applied in a variety of solutions and may be subjected to conventional pharmaceutical operations such as sterilization and/or may contain conventional adjuvants, such as preservatives, stabilizers, wetting agents, emulsifiers, buffers etc.

The GPBP inhibitors may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. The pharmaceutical compositions containing GPBP inhibitors may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the inhibitors in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the GPBP inhibitors in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

GPBP inhibitors may also be administered in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative, and flavoring and coloring agents. The GPBP inhibitor compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The GPBP inhibitor-containing compositions may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

The GPBP inhibitor-containing compositions of the present invention may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of GPBP inhibitors on the order of from about 0.01 mg to about 50 mg per kilogram of body weight per day, and more preferably between 0.1 mg to about 50 mg per kilogram of body weight per day, are useful in the treatment of the above-indicated conditions. The amount of GPBP inhibitor that may be combined with the carrier materials to produce a single dosage form will vary depending upon the host treated and the particular mode of administration. Dosage unit forms will generally contain between from about 1 mg to about 500 mg of inhibitor.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

In one embodiment of the methods of any therapeutic aspect of the invention, the GPBP inhibitor(s) may be co-administered with one or more other therapeutics for treating T2D and diabetic complications, including but not limited to insulin, metformin, sulfonylureas, alpha glucosidase inhibitors, thiazolidendiones, glucagon-like peptide-1 and analogs thereof, dipeptidyl peptidase-4 inhibitors, and angiotensin-converting enzyme inhibitors (ACEIs).

In another aspect, the present invention provides methods for diagnosing a pre-diabetic state comprising

(a) determining an amount of GPBP in a sample from a subject at risk of having a pre-diabetic state; and

(b) comparing the amount of GPBP in the sample to an amount of GPBP in a control sample;

wherein an increase in the amount of GPBP in the sample compared to the control sample indicates the presence of a pre-diabetic state in the subject.

As shown in the examples that follow, circulating levels GPBP were significantly increased in a normoglycemic irs2^{-/-} mice (model of T2D) as compared with wild-type (WT). However, in diabetic irs2^{-/-} mice, a marked reduction in circulating levels of GPBP was observed, demonstrating that up-regulation of GPBP occurs in the pre-diabetic state and predicts T2D development. Thus, GPBP is a biomarker of the pre-diabetic state that can be used clinically for the early detection of insulin resistance and propensity to develop T2D.

As used herein, a “pre-diabetic state” is the state in which some but not all of the diagnostic criteria for diabetes are met. Thus, the pre-diabetic state may comprise: 1) having impaired fasting glucose tolerance, which is a condition whereby the response of beta cells to an oral glucose challenge (OGT) is deficient or 2) having consistently elevated fasting glucose (IFG), which is a condition in which the fasting blood glucose is elevated above what is considered normal levels but is not high enough to be classified as diabetes mellitus. The pre-diabetic state may be associated with insulin resistance and increased risk of cardiovascular pathology. Individuals with a pre-diabetic state are at a relatively high risk of developing T2D. In a further aspect, the invention provides methods for diagnosing a propensity to develop T2D, comprising

(a) determining an amount of GPBP in a sample from a subject at risk of developing T2D; and

(b) comparing the amount of GPBP in the sample to an amount of GPBP in a control sample;

wherein an increase in the amount of GPBP in the sample compared to the control sample correlates with a propensity to develop T2D in the subject.

Such “increase” can be any amount of increase in GPBP relative to control (such as control sample from normal subject, or previously determined “normal” levels of GPBP in a control population), for example, 5%, 10%, 15%, 20%, 25%, 50%, 75%, 100%, or greater. In one embodiment, a normal value of GPBP as a reference for a standard curve is less than 10 ng/ml in plasma. In various embodiments, a normal GPBP range may be between ˜1 ng/ml-10 ng/ml in plasma. Thus, in one embodiment, subjects identified as having more than 10 ng/ml of GPBP in their plasma are treated according to the methods of the invention. Methods for determining the amount of circulating GPBP are known (see WO 2010/009856 and U.S. Pat. No. 7,935,492).

As used herein, a ‘propensity to develop T2D means that the subject has an increased risk of developing T2D compared to the general population.

In these embodiments, any suitable sample from a subject may be used as disclosed herein, including but not limited to a serum sample.

Subjects at risk of a pre-diabetic state and/or at risk of developing T2D include, but are not limited to, those subjects with one or more risk factors selected including, but not limited to obesity, a sedentary lifestyle, poor eating habits (ex: too much fat, not enough fiber, too many simple carbohydrates, etc.), family history of pre-diabetic state and/or T2D, age 50 or older, high blood pressure, high cholesterol, and history of gestational diabetes.

In one embodiment, the detecting comprises

(a) contacting a plasma sample from the subject with a GPBP binding molecule that binds to GPBP under conditions to promote selective binding of the GPBP-binding molecule to the GPBP;

(b) removing unbound plasma and/or GPBP-binding molecules; and

(c) detecting complex formation between the GPBP-binding molecule and the GPBP in the plasma sample, wherein complex formation provides a measure of GPBP in the sample.

A “GPBP binding molecule” is a peptide or nucleic acid molecule that binds selectively to GPBP, as opposed to one or more other biological molecules, structures, cells, tissues, etc. Exemplary embodiments of such GPBP binding molecules include but are not limited to antibodies, aptamers or substrates. As used herein, a “GPBP substrate” is a target of GPBP biological activity that binds to GPBP, or a fragment thereof that retains GPBP binding activity. Such GPBP substrates include, but are not limited to, I-20 (SEQ ID NO:9), GPBP-interacting proteins (GIPs) (SEQ ID NOS:14-18), myelin basic protein (MBP) and derivatives thereof (SEQ ID NOS:19-22), prion protein (PrP) (SEQ ID NO:23), α3NC1(SEQ ID NO:24), and Alzheimer's disease beta peptide (Aβ₁₋₄₂) (SEQ ID NO:25). Exemplary references demonstrating GPBP binding of these substrates can be found in U.S. Pat. Nos. 6,579,969; 7,147,855; and 7,326,768, incorporated by reference herein in their entirety.

A “plasma sample” means blood plasma, the liquid component of blood, and is prepared, for example, by centrifugation of whole blood to remove blood cells. As used herein, a plasma sample also includes a blood serum sample, in which blood clotting factors have been removed.

The plasma sample may be obtained from any suitable subject, preferably from a mammal that is at risk of T2D or pre-diabetes, including but not limited to a human, dog, cat, horse, or livestock (cow, sheep, etc.). In a most preferred embodiment, the plasma sample is obtained from a human subject.

The antibody can be any selective GPBP antibody, whether polyclonal, monoclonal, or humanized monoclonal as described above, although monoclonal antibodies are preferred.

Conditions suitable to promote binding of GPBP-binding molecules, such as antibodies, aptamers or substrates, to GPBP in the plasma samples can be determined by those of skill in the art based on the teachings herein and the examples provided below.

For example, antibody-antigen binding often depends on hydrophobic interactions (the so called hydrophobic bonds); thus, high salt concentrations, such as in the molar range can be used to reduce nonspecific binding and increase specific antigen-antibody binding. Optionally, further steps may be included to promote selectivity and specificity, including but not limited to one or more wash steps to remove unbound GPBP and/or GPBP-binding molecule, or unbound or weakly bound serum proteins; inhibitors of non-specific binding to reduce binding of high concentration serum proteins, control samples known to contain GPBP and/or negative controls known not to bind to GPBP, and/or inclusion of plasma samples known to not possess GPBP (ex: deleted for GPBP).

The methods can test for the presence of GPBP in the plasma sample by standard techniques including, but not limited to ELISA, immunoflourescence, and chromatography (for example, lateral flow assays where the antibody is immobilized on a surface and plasma proteins are labeled and allowed to flow over the surface under conditions suitable to permit binding of the antibody to GPBP in the plasma). In one embodiment, functional beads (Becton Dickinson technology) coupled to flow cytometry are used; this technique is an emerging method to measure the levels of proteins in biological fluid or cell/tissue extracts. Specifically, beads made of a fluorescence matrix are coated with one or more GPBP-specific antibodies, mixed with the plasma sample and further incubated with a detecting GPBP antibody labeled with a phycoerythrins. Finally, beads are analyzed by a flow cytometry program which selects the beads according matrix fluorescence emission and measurement of the level of the analyte through phycoerythrin emission. There are up to thirty different types of beads that can be simultaneously detected and discriminated by the cytometer. In another embodiment, microspheres (x MAP technology) coupled to flow cytometry or to CCD imaging based technology (multiplexing platforms) are used. Specifically, microspheres made of polystyrene and internally dyed with red and infrared fluorophores are coated with one or more GPBP-specific antibodies, mixed with the plasma sample, further incubated with a detecting GPBP antibody labeled with biotin and finally complexes are detected with streptavidin labeled with phycoerythrin. Beads are subsequently analyzed by multiplexing platforms which selects the beads according to fluorescence emission and measurement of the level of the analyte through phycoerythrin emission. There are up to five hundred different types of microspheres that can be simultaneously detected and discriminated by the multiplexing platforms. These methods couple high sensitivity and performance with versatility since a specific functional bead or a microsphere coated with GPBP-specific antibodies can be mixed with a distinct bead type coated with binding peptides for other analyte (i.e. autoantibodies) and simultaneously measured. The measurement of various analytes could enhance the potential of GPBP determination. In one embodiment, the techniques may determine only the presence or absence of the GPBP. Alternatively, the techniques may be quantitative, and provide information about the relative amount of the GPBP in the sample. For quantitative purposes, ELISAs are preferred.

Detection of immunocomplex formation can be accomplished by standard detection techniques. For example, detection of immunocomplexes can be accomplished by using labeled antibodies or secondary antibodies. Such methods, including the choice of label are known to those ordinarily skilled in the art. Alternatively, the antibodies can be coupled to a detectable substance. The term “coupled” is used to mean that the detectable substance is physically linked to the antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase. Examples of suitable prosthetic-group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of suitable radioactive material include ¹²⁵I, ¹³¹I, ³⁵S or ³H.

The methods comprise comparison of GPBP levels detected in a test plasma sample with a control, such as a control from a plasma sample known to have “normal” levels of GPBP or previously determined normal values for GPBP in plasma from the subject from whom the plasma is obtained. In various embodiments, the control provides a standard curve using recombinant GPBP or a reference value. In comparing the amount of GPBP in the plasma sample to a control, an increase in GPBP in the plasma sample relative to the control indicates the presence of a pre-diabetic state and/or a propensity to develop T2D.

In one embodiment, the method comprises detecting native circulating GPBP from human plasma. In another embodiment, the GPBP-binding molecule comprises an antibody, such as a monoclonal or polyclonal antibody.

### EXAMPLES

GPBP forms large multimeric aggregates whose specific kinase activity is much higher than that of aggregates of lower molecular weight (WO 2000/50607). A five-residue-long motif ²⁶⁰SHCIE²⁶⁴ (SEQ ID NO: 4) is the core of a region which is critical stabilizing native GPBP multimer assembly. An isolated peptide (Q2) representing ²⁶⁰SHCIE²⁶⁴(SEQ ID NO: 4) and flanking regions (LATLSHCIELMVKR) (SEQ ID NO: 8) efficiently inhibited GPBP kinase activity (WO 2004/070025). Organic compounds mimicking Q2 have been shown to inhibit GPBP kinase activity (WO 2011/054530 and U.S. Pat. No. 8,586,776). The anti-tumor agent doxorubicin has also been shown to inhibit GPBP kinase activity (WO2014/006020). Anti-GPBP monoclonal antibodies have also been shown to inhibit GPBP activity (WO 2012/113785).

T2D is associated with insulin resistance and β-cell failure. Insulin and IGF-I exert their biological activities via the phosphorylation and activation of insulin receptor substrate (IRS) proteins (Burks and White, 2001). Deletion of Irs2 in mice produces peripheral insulin resistance and a reduced in β-cell mass, leading to diabetes (Withers et al., 1998). Male Irs2-null mice develop hyperglycemia around 8 weeks of age and most die of diabetic complications before 16 weeks of age. The absence of IRS2 also causes moderate obesity in female mice (Burks et al., 2000). The diabetic phenotype displays a sexual dimorphism such that females develop diabetes more slowly, with many surviving up to 6 months of age (Garcia-Barrado et al., 2011).

### Experimental Methods

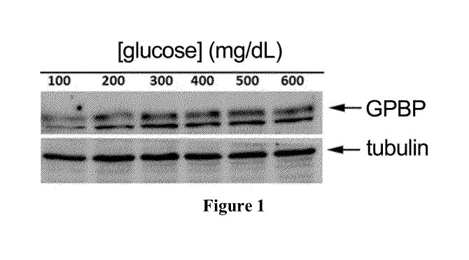

FIG. 1. Elevated extracellular glucose levels induces GPBP expression. C2C12 cells were differentiated for 5 days in DMEM (Dulbecco's modified Eagle's medium) supplemented with 2% horse serum and antibiotics. Cultures were washed with PBS (phosphate buffered saline) and incubated for another 24-hour period with DMEM supplemented with 2% horse serum containing glucose at the indicated concentrations. Cells were washed with PBS and lysed in 50 mM Tris-HCl pH 7.0, 150 mM NaCl, 1% Triton X-100, 1 mM PMSF (phenyl-methylsulfonyl fluoride), 10 mg/mL leupeptin for 30 min at 4° C. Lysates were cleared by centrifugation (16 000×g, 5 min, 4° C.), the total protein concentration of the supernatants was determined (Bio-Rad) and similar quantities (50 μg) of each extract were analyzed by Western blot with anti-GPBP monoclonal antibodies (mAb) N27 and anti-tubulin (Sigma), as elsewhere indicated.

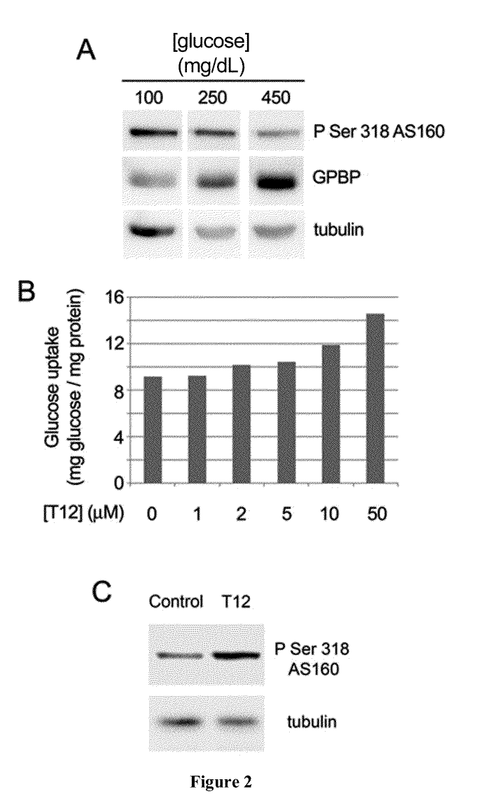

FIG. 2. GPBP kinase inhibition enhances glucose consumption under increased extracellular glucose concentrations. A, A549 cells were cultured in DMEM plus 10% fetal bovine serum and penicillin/streptomycin with the indicated concentrations of glucose during 24 h. Then cells were lysed in 25 mM Tris-HCl pH 7.5, 150 mM NaCl, 1% Triton X-100, 0.1% SDS, 1 mM PMSF, 10 μg/mL leupeptin, 20 mM NaF at 4° C. during 30 min. Lysates were cleared by centrifugation at 16,000×g for 5 min (4° C.) and the protein concentration of supernatants was determined (Bio-Rad). Similar amounts (50 μg) of each lysate were analyzed by Western blot for detection of AS160 protein phosphorylated at Ser 318 (P Ser 318 AS160), GPBP and tubulin, using primary antibodies obtained from Cell Signaling Technology, Fibrostatin SL and Sigma, respectively, and HRP-conjugated anti-rabbit IgG and anti-mouse IgG secondary antibodies from Promega. Development was performed with Amersham ECL Prime Western Blotting Detection Reagent (GE Healthcare Lifesciences) and image acquisition with an ImageQuant LAS 4000 Mini device (GE Healthcare Lifesciences). B, A549 cells were cultured in DMEM: F-12 (315 mg glucose/dL) supplemented with 10% fetal bovine serum and penicillin/streptomycin with the indicated concentrations of T12 during 14 h. After incubations cells were lysed as in A and protein concentrations in supernatants were similarly assessed. Glucose concentrations in the media were determined at the beginning and at the end of the incubation period with a Glucocard G+ meter (Arkray Factory, Inc.), and glucose uptake by cells was calculated using the protein concentrations of the corresponding lysates for normalization purposes. Similar results were obtained with doxorubicin (0.5 μM) (data not shown), a different GPBP kinase inhibitor (WO/2014/006020). C, A549 cells were incubated in DMEM: F-12 (315 mg glucose/dL) plus 10% fetal bovine serum and penicillin/streptomycin during 20 h in absence (Control) or presence of T12 (50 μM), and lysed as in A. Similar amounts (50 μg) of each lysate were analyzed by Western blot for detection of AS160 protein phosphorylated at Ser 318 (P Ser 318 AS160) or of tubulin as in A.

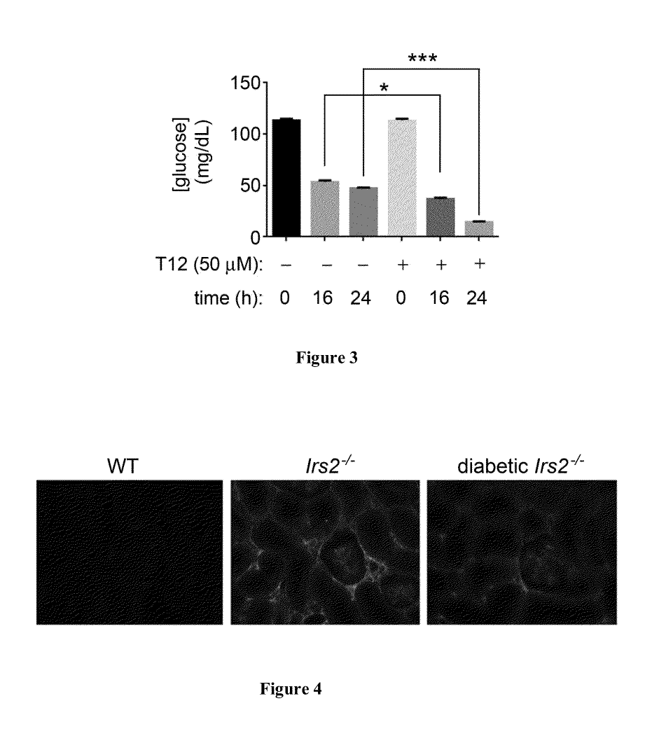

FIG. 3. GPBP kinase inhibition also enhances glucose consumption under physiological extracellular glucose concentrations. C2C12 mouse myoblasts were differentiated to myotubes in DMEM supplemented with 2% horse serum and penicillin/streptomycin during 10 days, and then incubated in DMEM plus 0.25% horse serum with an initial glucose concentration of 115 mg/dL, in absence or presence of T12 (50 μM) during the indicated times. Glucose concentrations in media were determined with a Glucocard™ G+ meter (Arkray Factory, Inc.). Shown are means±SEM (n=2). Differences between the indicated groups were statistically significant according to t-Student test analysis. *P=0.0152; ***P=0.0005.

FIG. 4. Irs2^{-/-} mice displayed increased collagen IV expression. Kidneys were collected from male wild type (WT, n=9), normoglycemic Irs2^{-/-} (Irs2^{-/-}, n=9) and diabetic Irs2^{-/-} (n=9) mice at 8-12 weeks of age and embedded in paraffin for immunohistochemical analysis of sections with anti-type IV collagen antibodies from EMD Millipore. Mice were considered diabetic when serum glucose levels under fasting were greater than 120 mg/dL. In the composite the level of collagen IV expression is represented in a black (no expression) and white (expression) color range. Shown are representative images. Original magnification: ×400.

FIG. 5. Irs2^{-/-} mice displayed increased COL4A3BP expression. Kidneys were collected from male wild type (WT, n=9), normoglycemic Irs2^{-/-} (Irs2^{-/-}, n=9) and diabetic Irs2^{-/-} (n=9) mice at 8-12 weeks of age and embedded in paraffin for immunohistochemical analysis of sections with anti-GPBP mAb N26 from Fibrostatin SL. Mice were considered diabetic when serum glucose levels under fasting were greater than 120 mg/dL. In the composite the level of GPBP expression is represented in a black (no expression) and white (expression) color range. Shown are representative images. Original magnification: ×400.

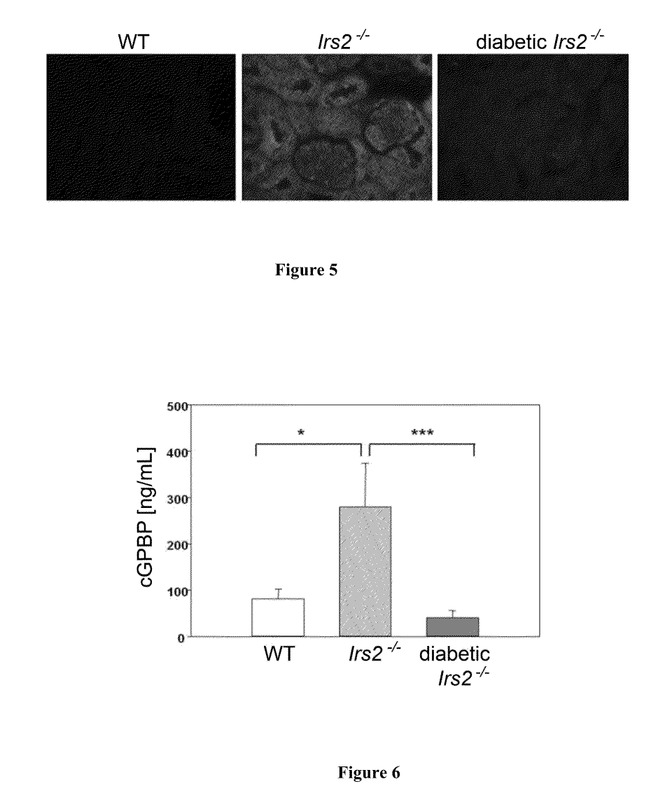

FIG. 6. Circulating GPBP levels are augmented in normoglycemic Irs2-deficient mice. Blood samples were collected from wild type (WT, n=12), normoglycemic Irs2^{-/-} (Irs2^{-/-}, n=12) and diabetic Irs2^{-/-} (n=19) mice at the time of sacrifice (8-12 weeks of age) and circulating GPBP levels were measured using mAb N26 as capturing and mAb N27 as detecting antibodies in a prototype sandwich ELISA essentially as described in WO 2010/009856. Mice were considered diabetic when serum glucose levels under fasting were greater than 120 mg/dL. *P<0.05, ***P<0.001.

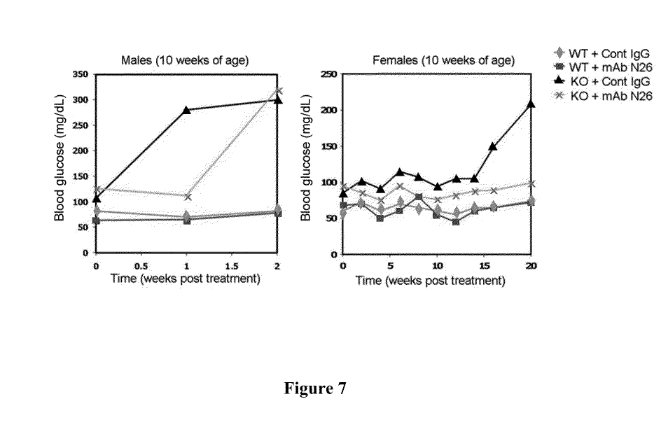

FIG. 7. Effect of anti-GPBP treatment on blood glucose in 10-week old mice. Different groups of mice received weekly intraperitoneal injections of anti-GPBP mAb N26 or mouse IgG (Sigma) at 1 μg/g of body weight, and fasting blood glucose (tail vein) was monitored weekly using a glucometer. Males. Wild type treated (WT+mAb N26, n=2), wild type control (WT+Cont IgG, n=2), Irs2^{-/-} treated (KO+mAb 26, n=2), Irs2^{-/-} control (KO+Cont IgG, n=2). Females. Wild type treated (n=2), wild type control (n=2), Irs2^{-/-} treated (n=3), irs2^{-/-} control (n=2).

### Results and Discussion

In differentiated myoblasts, downregulation of GPBP expression (i.e. Gpbp^{-/-}) associated with increased activation of Akt (Revert-Ros et al., 2011) revealing that cytosolic GPBP downregulates Akt activation and renders the cells with less capacity to uptake glucose in response to insulin (insulin resistance). Consistently, increasing extracellular glucose induced GPBP expression in differientated myoblast (FIG. 1) suggesting that intracellular levels of GPBP increase in response to increasing extracellular glucose concentrations (hyperglycaemia) perhaps in an attempt of the cells to prevent deleterious effects of elevated intracellular glucose concentrations. Accordingly, when exposing A549 cultures to increasing concentration of extracellular glucose we observed a progressive increased expression of GPBP which associated with a lower phosphorylation state of Akt-substrate 160 (AS160), the hallmark for induction of glucose uptake (FIG. 2A). Moreover, increasing concentrations of GPBP kinase inhibitor compound T12 (3-[4″-methoxy-3,2′-dimethyl-(1,1′;4′,1″)terphenyl-2″-yl]propionic acid (WO 2011/054530 and U.S. Pat. No: 8,586,776); progressively enhanced glucose uptake in A549 cells cultured under conditions representing the hyperglycaemic state of T2D (FIG. 2B). All of which suggested that cytosolic GPBP kinase inhibition by compound T12 induced glucose uptake through Akt disinhibition and subsequent phosphorylation of AS160. Accordingly, analysis of the corresponding cell cultures lysates by Western blot revealed that compound T12 induced phosphorylation of AS160 (FIG. 2C) which in turn is expected to promote translocation of glucose transporters GLUT1/GLUT4 to the plasma membrane and glucose uptake (Sano et al., 2003). Collectively, the evidence suggests that enhanced cytosolic kinase activity of GPBP associated with hyperglycemia mediates at least in part peripheral insulin resistance. Similar conclusions were obtained when treating cell cultures with doxorubicin (0.5 μM) (data not shown), an additional structurally unrelated GPBP kinase inhibitor (WO2014/006020). Further, the role of GPBP kinase activity was assessed in physiological glucose concentrations and shown that compound T12 also reduced the extracellular glucose levels in a concentration-dependent manner (FIG. 3), suggesting that cytosolic GPBP kinase activity down regulates insulin-dependent signaling in both normo-glycemic and diabetic conditions.

To investigate the role of GPBP in T2D we have first analyzed the expression of both GPBP and collagen IV in the kidneys of Irs2^{-/-} mice. Diabetic nephropathy is characterized by accumulation of mesangial matrix and thickening of the basement membrane in the glomeruli as well as renal tubular hypertrophy. These abnormalities are associated with renal overproduction of extracellular matrix proteins such as collagen IV. Consistent with this, clinical studies have reported a progressive increase in urinary collagen IV excretion in diabetic patients (Cawood et al., 2010). Consequently, a high level of urinary collagen IV has been proposed as a marker of development and progression of early diabetic kidney disease. In assessing diabetes in the Irs2^{-/-} mouse model, we apply the same criteria for blood glucose values as in humans; fasting levels >120 mg/dL are considered diabetic. We observed that mice with severe hyperglycemia often presented proteinuria (>100 mg/dL), suggesting a deterioration of renal function associated with high glucose levels. Thus, we used immunohistochemistry to assess collagen IV expression in kidneys obtained from wild type (WT) Irs2^{+/+} mice, normoglycemic Irs2^{-/-} mice and diabetic Irs2^{-/-} mice. We observed that the expression of collagen IV was increased in both groups of Irs2^{-/-} mice in comparison to WT control kidney but being more expressed in normoglycemic than in diabetic Irs2^{-/-} mice (FIG. 4). Given the role of GPBP in regulating collagen IV supramolecular organization and triggering glomerulosclerosis when overexpressed (Revert et al., 2007), we examined GPBP expression in kidney sections of control and Irs2^{-/-} mice. Interestingly, GPBP expression paralleled collagen IV expression and thus GPBP was overexpressed in the kidneys of Irs2^{-/-} mice as compared with WT controls and still more expressed in normoglycemic than in diabetic Irs2^{-/-} mice (FIG. 5). Thus, these findings suggest that pro-inflammatory cytokines associated with the pre-diabetic state in Irs2^{-/-} mice may up regulate GPBP expression which then declines to a more basal level in mice with overt diabetes (inflammatory stage). These results are consistent with GPBP being a pre-pro-inflammatory circulating factor (WO 2012/113785) that also plays a role in T2D of Irs2^{-/-} mouse model.

To further assess this possibility we used a previously developed ELISA for detection of circulating GPBP (WO 2010/009856 and U.S. Pat. No. 7,935,492) to measure the levels of GPBP in serum samples obtained from control mice and the two groups of Irs2^{-/-} (normoglycemic and diabetic). Consistent with the results of the immunostainings, circulating levels of GPBP were significantly increased in normoglycemic Irs2^{-/-} mice as compared with WT (FIG. 6). However, in diabetic Irs2^{-/-} mice, we observed a marked reduction in circulating levels of GPBP. These results suggest that up regulation of GPBP occurs in the pre-diabetic state likely in parallel with pro-inflammatory cytokines Hence, given the availability of a sensitive ELISA for detecting GPBP in serum, our observations in the Irs2^{-/-} model indicate that GPBP is a potential novel biomarker of the pre-diabetic state that could be used clinically for the early detection of insulin resistance and diabetes.

Deletion of Irs2 produces diabetes in mice owing to peripheral insulin resistance and a reduction in pancreatic β-cell mass (Withers et al., 1998). However, this diabetic phenotype displays a sexual dimorphism; male Irs2^{-/-} mice often die of diabetic complications by 12 weeks of age but female Irs2^{-/-} develop a milder form of diabetes and many live up to 6 months (Garcia-Barrado, 2011). Additionally, female Irs2^{-/-} mice display hyperleptinemia and develop moderate obesity, in contrast to male Irs2^{-/-} which are often leaner than control mice (Burks et al., 2000).

Our observation that GPBP and collagen IV are up regulated in normoglycemic Irs2^{-/-} mice suggests that this kinase may be involved in the progression from the pre-diabetic to the diabetic state. If indeed GPBP has a role in this pathology, GPBP inhibition would be expected to delay or prevent development of diabetes in pre-diabetic models. To test this possibility, normoglycemic Irs2^{-/-} mice were treated with N26, GPBP-specific antibodies which inhibit the binding of GPBP to the α3NC1 domain of collagen IV (WO 2012/113785). Glucose levels were then monitored as an indicator of metabolic status. Given the sexual dimorphism present in this model of diabetes, we assessed anti-GPBP in both males and females. In this study, treatment was begun when mice were 10 weeks of age and N26 was administered at 1 μg/g of body weight once weekly via an intraperitoneal injection. Fasting glucose levels in WT females and males treated with anti-GPBP were similar to those treated with a control IgG, demonstrating that the anti-GPBP treatment did not alter basal glucose levels (FIG. 7). Although treatment was able to maintain glycaemia at normal levels in Irs2^{-/-} males a week after treatment initiation, hyperglycemia proceeded rapidly during the second week and reached similar values regardless of whether mice received injections of N26 or not; and thus all Irs2^{-/-} males died of diabetic complications within a month of the study onset. In contrast, female Irs2^{-/-} treated with anti-GPBP therapy lived for up to 7 months with normal glucose levels whereas the female Irs2^{-/-} treated with a control IgG progressively developed hyperglycaemia.

Collectively, the data support that pre-pro-inflammatory extracellular GPBP (WO 2012/113785) mediates pathogenesis in the Irs2^{-/-} model for T2D. This also supports the notion that increased circulating levels of GPBP are diagnostic of incoming inflammation that mediates hyperglycaemia and T2D. Accordingly, early administration of GPBP blocking antibodies (pre-diabetic) delayed hyperglycaemia onset in males and precluded hyperglycaemia in females. Thus, we uncovered extracellular GPBP to be a new potential biomarker for detecting pre-diabetic stage as well as a new potential therapeutic target for treating T2D. Moreover, anti-GPBP antibodies emerge as a double-edge sword against T2D detecting patients that will develop T2D and as a biological drug to treat these patients.

Of special interest was to observe that individual administration of three unrelated down regulators of GPBP (i.e. T12, doxorubicin and N26) in different experimental models (i.e. myocyte, lung adenocarcinoma A549 cell line and Irs2^{-/-} mouse) reduced extracellular glucose concentrations. The evidence also supports that reduction of extracellular glucose is accomplished at least in part through phosphorylation of the cytosolic protein AS160 leading to enhanced glucose uptake. This observation provides further independent evidence for extracellular GPBP to regulate intracellular GPBP activity mediating pathogenesis in the Irs2^{-/-} mouse model for T2D.

### REFERENCES

Burks D J, Font de Mora J, Schubert M, Withers D J, Myers M J, Towery H, Altamuro S A, Flint C A, White M F. 2000. IRS-2 pathways integrate female reproduction and energy homeostasis. Nature 407:377-82.
Burks D J, White M F. 2001. The role of IRS proteins in beta cell physiology. Diabetes 456: 120-125.
Cawood T J, Bashir M, Brady J, Murray B, Murray P T, O'Shea D. 2010. Urinary collagen IV and πGST: potential biomarkers for detecting localized kidney injury in diabetes—a pilot study. Am. J. Nephrol. 32:219-25.
Garcia-Barrado M J, Iglesias-Osma M C, Moreno-Viedma V, Pastor Mansilla M F, Gonzalez S S, Carretero J, Moratinos J, Burks D J. 2011. Differential Sensitivity To Adrenergic Stimulation Underlies The Sexual Dimorphism In The Development Of Diabetes Caused By Irs-2 Deficiency. Biochem Pharmacol. 81:279-88.
Revert F, Merino R, Monteagudo C, Macias J, Peydró A, Alcácer J, Muniesa P, Marquina R, Blanco M, Iglesias M, Revert-Ros F, Merino J, Saus J. 2007. Increased Goodpasture antigen-binding protein expression induces type IV collagen disorganization and deposit of immunoglobulin A in glomerular basement membrane. Am. J. Pathol. 171:1419-30.
Revert-Ros F, López-Pascual E, Granero-Moltó F, Macias J, Breyer R, Zent R, Hudson B G, Saadeddin A, Revert F, Blasco R, Navarro C, Burks D, Saus J. Goodpasture antigen-binding protein (GPBP) directs myofibril formation: identification of intracellular downstream effector 130-kDa GPBP-interacting protein (GIP130). J Biol Chem. 286:35030-43.
Sano H, Kane S, Sano E, Mîinea C P, Asara J M, Lane W S, Garner C W, Lienhard G E. 2003. Insulin-stimulated phosphorylation of a Rab GTPase-activating protein regulates GLUT4 translocation. J Biol Chem. 278:14599-602.

Withers D J, Gutierrez J S, Towery H, Burks D J, Ren J M, Previs S, Zhang Y, Bernal D, Pons S, Shulman G I, Bonner-Weir S, White M F. 1998. Disruption of IRS-2 causes type 2 diabetes in mice. Nature. 391:900-4.

We claim:

1. A method for treating type 2 diabetes, comprising administering to a subject in need thereof an amount effective of a Goodpasture Antigen Binding Protein (GPBP) inhibitor to treat type 2 diabetes.

2. A method for limiting development of type 2 diabetes, comprising administering to a subject in need thereof an amount effective of a GPBP inhibitor to limit development of type 2 diabetes.

3. A method for treating a pre-diabetic state, comprising administering to a subject in need thereof an amount effective of a GPBP inhibitor to treat the pre-diabetic state.

4. The method of claim 1, wherein the subject has increased circulating GPBP relative to control.

5. The method of claim 2, wherein the subject has a pre-diabetic state.

6. The method of claim 1, wherein the GPBP inhibitor comprises an anti-GPBP monoclonal antibody.

7. The method of claim 6, wherein the anti-GPBP monoclonal antibody is a humanized monoclonal antibody.

8. The method of claim 1, wherein the GPBP inhibitor comprises a compound of formula: 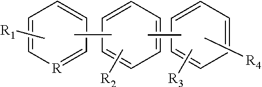
or a pharmaceutically acceptable salt thereof, wherein:
R is selected from N and CR₅;
R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), —(CH₂)₁₋₅—C(O)NH₂, (aryl)C₂-C₆ alkyl, and (heteroaryl)C₁-C₆ alkyl;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH₂, (aryl)C₁-C₆ alkyl, or (heteroaryl)C₁-C₆ alkyl;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH2, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), (aryl)C₁-C₆ alkyl, or (heteroaryl)C₁-C₆ alkyl; and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH2, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, —O(CH₂)₁₋₅—C(O)(C₁-C₆ alkoxy), (aryl)C₁-C₆ alkyl, or (heteroaryl)C₁-C₆ alkyl.

9. The method of claim 1, wherein the GPBP inhibitor comprises a compound having the formula: 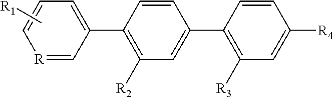
or a pharmaceutically acceptable salt thereof, wherein:
R is selected from N and CR₅;
R₅ is selected from the group consisting of hydrogen, halogen, cyano, nitro, hydroxy, C₁-C₆ alkyl, C₂-C₆ alkenyl, C₂-C₆ alkynyl, halo(C₁-C₆ alkyl), C₁-C₆ alkoxy, halo(C₁-C₆ alkoxy), amino, (C₁-C₆ alkyl)amino, di(C₁-C₆ alkyl)amino, hydroxy(C₁-C₆ alkyl), (C₁-C₆ alkoxy)C₁-C₆ alkyl, amino(C₁-C₆ alkyl), sulfanyl(C₁-C₆ alkyl), (C₁-C₆ alkyl)sulfanyl(C₁-C₆ alkyl), —(CH₂)₁₋₅—C(O)(C₁-C₆ alkoxy), and —(CH₂)₁₋₅—C(O)NH₂;
R₁ is hydrogen, halogen, hydroxy, C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, amino(C₁-C₆ alkyl), sulfanyl(C₁-C₆ alkyl), or (C₁-C₆ alkyl)sulfanyl(C₁-C₆ alkyl);
R₂ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₀-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), or —(CH₂)₁₋₅—C(O)NH₂;
R₃ is C₁-C₆ alkyl, halo(C₁-C₆ alkyl), C₁-C₆ alkoxy, halo(C₁-C₆ alkoxy), hydroxy(C₁-C₆ alkyl), (C₁-C₆ alkoxy)C₁-C₆ alkyl, formyl(C₁-C₆ alkyl), amino(C₁-C₆ alkyl), sulfanyl(C₁-C₆ alkyl), (C₁-C₆ alkyl)sulfanyl(C₁-C₆ alkyl), —(CH₂)₁₋₅—C(O)OH, —(CH₂)₁₋₅—C(O)(C₁-C₆ alkoxy), —(CH₂)₁₋₅—C(O)NH2, —(CH₂)₁₋₅—C(O)NH(C₁-C₆ alkyl), —(CH₂)₁₋₅—C(O)N(C₁-C₆ alkyl)₂, —CH═CH—C(O)OH, or —CH═CH—C(O)(C₁-C₆ alkoxy); and
R₄ is hydroxy, halogen, C₁-C₆ alkyl, C₁-C₆ alkoxy, halo(C₁-C₆ alkoxy), benzyloxy, —(CH₂)₁₋₅—C(O)OH, —(CH₂)₁₋₅—C(O)(C₁-C₆ alkoxy), —(CH₂)₁₋₅—C(O)NH₂, —(CH₂)₁₋₅—C(O)NH(C₁-C₆ alkyl), —(CH₂)₁₋₅—C(O)N(C₁-C₆ alkyl)₂, —CH═CH—C(O)OH, —CH═CH—C(O)(C₁-C₆ alkoxy), —O(CH₂)₁₋₅—C(O)OH, or —O(CH₂)₁₋₅—C(O)(C₁-C₆ alkoxy).

10. The method of claim 1, wherein the GPBP inhibitor comprises 3-[4″-methoxy-3,2′-dimethyl-(1,1′;4′,1″)terphenyl-2″-yl]propionic acid; 3-[2′-methyl-4″-methoxy-3-(trifluoromethyl)-(1,1′;4′,1″)terphenyl-2″-yl]propionic acid; combinations thereof, or a pharmaceutically acceptable salt thereof.

11. The method of claim 1, wherein the GPBP inhibitor comprises (3-[4″-methoxy-3,2′-dimethyl-(1,1′;4′,1″)terphenyl-2″-yl]propionic acid, or a pharmaceutically acceptable salt thereof.

12. The method of claim 1, wherein the GPBP inhibitor comprises one or more inhibitors selected from the group consisting of:
(a) an antibody or aptamer selective for GPBP;
(c) an inhibitory nucleic acid selective for GPBP mRNA;
(d) a peptide comprising an amino acid sequence of general formula X1-SHCIX2-X3 (SEQ ID NO: 1)
wherein X1 is 0-10 amino acids of the sequence ATTAGILATL (SEQ ID NO: 2);
X2 is E or Q; and
X3 is 0-10 amino acids of the sequence LMVKREDSWQ (SEQ ID NO: 3);
(e) a peptide comprising an amino acid sequence selected from the group consisting of SHCIE (SEQ ID NO: 4), SHCIQ (SEQ ID NO: 5), ILATLSHCIELMVKR (SEQ ID NO: 6), ILATLSHCIQLMVKR (SEQ ID NO: 7), and LATLSHCIELMVKR (SEQ ID NO: 8); and
(f) I-20:
(SEQ ID NO: 9)
RDEVIGILKAEKMDLALLEAQYGFVTPKKVLEALQRDAFQAKSTPWQEDI
YEKPMNELDKVVEKHKESYRRILGQLLVAEKSRRQTILELEEEKRKHKEY
MEKSDEFICLLEQECERLKKLIDQEIKSQEEKEQEKEKRVTTLKEELTKL
KSFALMVVDEQQRLTAQLTLQRQKIQELTTNAKETHTKLALAEARVQEEE
QKATRLEKELQTQTTKFHQDQDTIMAKLTNEDSQNRQLQQKLAALSRQID
ELEETNRSLRKAEEE.

13. A method for diagnosing a pre-diabetic state comprising
(a) determining an amount of GPBP in a sample from a subject at risk of having a pre-diabetic state; and
(b) comparing the amount of GPBP in the sample to an amount of GPBP in a control sample;
wherein an increase in the amount of GPBP in the sample compared to the control sample indicates the presence of a pre-diabetic state in the subject.

14. A method for diagnosing a propensity to develop type 2 diabetes, comprising
(a) determining an amount of GPBP in a sample from a subject at risk of developing type 2 diabetes; and
(b) comparing the amount of GPBP in the sample to an amount of GPBP in a control sample;
wherein an increase in the amount of GPBP in the sample compared to the control sample indicates a propensity to develop type 2 diabetes in the subject.

15. The method of claim 13, wherein the detecting comprises
(a) contacting a plasma sample from the subject with a GPBP binding molecule that binds to GPBP under conditions to promote selective binding of the GPBP binding molecule to the GPBP;
(b) removing unbound plasma and/or GPBP binding molecules; and
(c) detecting complex formation between the GPBP binding molecule and the GPBP in the plasma sample, wherein complex formation provides a measure of GPBP in the sample.

16. The method of claim 15, wherein the method comprises detecting native circulating GPBP from human plasma.

17. The method of claim 13, wherein the GPBP binding molecule comprises an antibody.

18. The method of claim 17, wherein the antibody is a monoclonal antibody.

19. The method of claim 13, wherein the GPBP is GPBP-1.
